(19) United States
(12) Patent Application Publication
(10) Pub. No.: US 2006/0019272 A1
(43) Pub. Date: Jan. 26, 2006

(54) Diagnosis of disease and monitoring of therapy using gene expression analysis of peripheral blood cells

(75) Inventors: Mark W. Geraci, Aurora, CO (US); Todd M. Bull, Denver, CO (US); Norbert F. Voelkel, Denver, CO (US); Christopher D. Coldren, Denver, CO (US)

Correspondence Address: Sheridan Ross P.C., Suite 1200, 1560 Broadway, Denver, CO, 80202-5141, US

(73) Assignee: The Regents of the University of Colorado, Boulder, CO (US)

(21) Appl. No.: 11/122,329

(22) Filed: May 3, 2005

Related U.S. Application Data
(60) Provisional application No. 60/568,129, filed on May 3, 2004.

(51) Int. Cl. C12Q 1/68

(52) U.S. Cl. 435/6

(57) ABSTRACT

Disclosed are methods to diagnose a patient that has a pulmonary disease, and particularly, pulmonary arterial hypertension, using biomarkers that are differentially regulated in the peripheral blood cells of patients with such disease as compared to individuals that do not have the disease. Also disclosed are methods to diagnose a patient that has idiopathic pulmonary arterial hypertension as compared to pulmonary arterial hypertension associated with secondary causes. Pluralities of nucleotides and antibodies useful in the invention are described. Methods of identifying compounds with the potential to treat pulmonary arterial hypertension (PAH) are also described.

### CROSS-REFERENCE TO RELATED APPLICATIONS

This application claims the benefit of priority under 35 U.S.C. § 119(e) from U.S. Provisional Application Ser. No. 60/568,129, filed May 3, 2004. The entire disclosure of U.S. Provisional Application Ser. No. 60/568,129 is incorporated herein by reference.

### GOVERNMENT SUPPORT

This invention was made with government support under Grant Nos. HL66254 and HL72340, each awarded by the National Institutes of Health. The government has certain rights to this invention.

### REFERENCE TO SEQUENCE LISTING

This application contains a Sequence Listing submitted on a compact disc, in duplicate. Each of the two compact discs, which are identical to each other pursuant to 37 CFR § 1.52(e)(4), contains the following file: “Sequence Listing”, having a size in bytes of 480 KB, recorded on 3 May 2005. The information contained on the compact disc is hereby incorporated by reference in its entirety pursuant to 37 CFR § 1.77(b)(4).

### FIELD OF THE INVENTION

This invention is generally related to diagnostic and prognostic assays and kits for pulmonary arterial hypertension (PAH) and other lung disorders. The invention includes the identification and use of biomarkers that are differentially expressed in PAH versus normal controls, as well as biomarkers that are differentially expressed between patients with idiopathic PHA and PAH due to secondary causes.

### BACKGROUND OF THE INVENTION

Pulmonary arterial hypertension (PAH) is characterized by a pressure elevation in the pre-capillary pulmonary vasculature of the lung. PAH is associated with a number of devastating diseases and the severe elevation in pulmonary arterial (PA) pressure can eventually lead to right heart failure and death (1-4). The PA pressure elevation observed in this group of diseases is associated with micro-vascular remodeling and endothelial cell proliferation which includes the development of plexiform lesions (5-7). It is currently not possible to distinguish between the various forms of severe PAH by examination of the lung histology alone, as the pulmonary vascular pathologic alterations are identical (1). It is hypothesized that the development of PAH requires first a genetic susceptibility followed by one or several secondary trigger factors such as a viral infection or drug exposure (8,9). While a number of promoting events are currently recognized, the individual's genetic susceptibility and the interaction of the genotype with the promoting factor or factors remain areas of active research.

It is currently hypothesized that inflammation plays an important role in the development of some or all forms of severe pulmonary hypertension (PH) (10-12). PAH is a recognized complication of a number of systemic inflammatory conditions such as scleroderma and systemic lupus erythematosus (SLE) (13). Mononuclear inflammatory cells surround the plexiform lesions in patients with scleroderma-related PAH and primary pulmonary hypertension (PPH; also known as idiopathic pulmonary arterial hypertension, or IPAH) (14, 15). Plasma levels of inflammatory markers are elevated in patients with IPAH compared to normal controls (16, 17). In concert with potential inflammatory mechanisms there exits evidence that immunologic abnormalities may be also be associated with the development of PAH. Patients with HIV-1 infection or with the POEMS syndrome (polyneuropathy, organomegaly, endocrinopathy, M protein and skin changes) are known to develop severe pulmonary hypertension (8; 18). A significant number of patients diagnosed with IPAH have evidence of an autoimmune disorder with inflammation. These abnormalities include the presence of antinuclear antibodies, increased serum levels of pro-inflammatory cytokines such as IL-1 and IL-6, increased incidence of certain MHC class II molecules, and increased pulmonary expression of platelet-derived growth factor and macrophage inflammatory protein-α (MIP-1α) (10; 19-21). Patients with PAH also have a higher prevalence of autoimmune thyroid disease when compared to the general population and most recently, the present inventors have reported a new association between human herpesvirus-8 infection and IPAH (22-24).

The pathogenesis of severe PAH is complex and it is likely that multiple modulating genes and environmental factors are involved. Such complexity lends itself to the use of microarray technology, which allows the efficient and accurate simultaneous expression measurement of thousands of genes (25). This technology has been most successfully employed in the investigation of cancer, including hematologic malignancies and in the classification of histologically indistinct tumor types with different natural histories (26-28). Microarray expression profiles have also been used to assess a tumor's metastatic potential, tissue of origin and susceptibility to chemotherapeutic agents (28-30). A significant challenge to the study of gene expression is the collection of biological material of sufficient homogeneity, quantity and quality for microarray study. Biopsy specimens from patients with early-stage disease tend to be small, and routine histological preservation (formalin fixation) generally prohibits quality ribonucleic acid extraction. Furthermore, in diseases such as PAH, a lung biopsy is relatively contraindicated due the high associated morbidity and mortality of the procedure.

Therefore, there is a need in the art for robust, diagnostic and prognostic tests for severe pulmonary hypertension that are safe and relatively non-invasive for the patient.

### SUMMARY OF THE INVENTION

One embodiment of the present invention relates to a method to diagnose pulmonary arterial hypertension (PAH) or a predisposition to develop PAH. The method includes the steps of: (a) detecting in a sample of peripheral blood cells from a patient to be tested the level of expression of at least one biomarker chosen from a panel of biomarkers whose expression in peripheral blood cells has been associated with PAH as measured by either upregulation or downregulation of biomarker expression in peripheral blood cells from patients with PAH as compared to the level of expression of the biomarkers in peripheral blood cells from normal controls; (b) comparing the level of expression of the biomarker or biomarkers detected in the patient sample to a level of expression of the biomarker or biomarkers that has been associated with PAH and a level of expression of the biomarker or biomarkers that has been associated with normal controls; and (c) diagnosing PAH in the patient if the expression level of the biomarker or biomarkers in the patient sample is statistically more similar to the expression level of the biomarker or biomarkers that has been associated with PAH than the expression level of the biomarker or biomarkers that has been associated with the normal controls.

In one aspect of this embodiment of the invention, the panel of biomarkers in (a) is identified by a method comprising; (1) comparing the expression level of at least one biomarker in peripheral blood cells from patients that have PAH to the level of expression of the biomarker in peripheral blood cells from normal controls that do not have PAH; and (2) identifying a biomarker or biomarkers having a level of expression in peripheral blood cells from patients with PAH that is statistically significantly different than the level of expression of the biomarker or biomarkers in the peripheral blood cells from the normal controls, as being a biomarker for use in a panel of biomarkers to diagnose PAH.

In one aspect of this embodiment, step (a) comprises detecting in the patient sample the expression of at least one gene chosen from a gene comprising, or expressing a transcript comprising, a nucleic acid sequence selected from the group consisting of SEQ ID NOs:1-101; step (b) comprises comparing the level of expression of the gene or genes detected in the patient sample to a level of expression of the gene or genes that has been associated with PAH and to a level of expression of the gene or genes that has been associated with normal controls; and step (c) comprises diagnosing PAH in the patient, if the expression of the gene or genes in the patient sample is statistically more similar to the expression level of the gene or genes that has been associated with PAH than with normal controls. Other aspects of the invention include detecting the expression of at least 2 genes, at least 5 genes, at least 10 genes, at least 25 genes, at least 50 genes, at least 75 genes, at least 100 genes, at least 125 genes, up to detection of all of the genes representing the panel of biomarkers, or each of SEQ ID NOs:1-101.

Various techniques can be used to detect the expression of the gene or genes including, but not limited to, measuring amounts of transcripts of the gene in the patient peripheral blood cells, detecting hybridization of at least a portion of the gene or a transcript thereof to a nucleic acid molecule comprising a portion of the gene or a transcript thereof in a nucleic acid array, or using quantitative polymerase chain reaction (q-PCR). In one embodiment, expression of the gene is detected by detecting the production of a protein encoded by the gene.

In one aspect of this embodiment of the invention, the method also or further includes determining if the patient has idiopathic pulmonary arterial hypertension (IPAH) or secondary pulmonary arterial hypertension (s-PAH). The step of determining includes: (a) comparing the level of expression of at least one gene chosen from a gene comprising, or expressing a transcript comprising, a nucleic acid sequence selected from: SEQ ID NO:84, SEQ ID NOs:102-128; (b) comparing the level of expression of the gene or genes detected in the patient sample to a level of expression of the gene or genes that has been associated with IPAH and to a level of expression of the gene or genes that has been associated with s-PAH; and (c) diagnosing IPAH in the patient, if the expression of the gene or genes in the patient sample is statistically more similar to the expression level of the gene or genes that has been associated with IPAH than with s-PAH, or diagnosing S-PAH in the patient, if the expression of the gene or genes in the patient sample is statistically more similar to the expression level of the gene or genes that has been associated with s-PAH than with IPAH.

In one aspect of this embodiment of the invention, the level of expression of the gene or genes that has been associated with PAH and the level of expression of the gene or genes that has been associated with normal controls has been predetermined.

Another embodiment of the present invention relates to a plurality of polynucleotides for the detection of the expression of genes that indicate a diagnosis of pulmonary arterial hypertension (PAH) in a patient, wherein the plurality of polynucleotides consists of at least two polynucleotides, wherein each polynucleotide is at least 5 nucleotides in length, and wherein each polynucleotide is complementary to an RNA transcript, or nucleotide derived therefrom, of a gene that is regulated differently in peripheral blood cells of patients with PAH as compared to peripheral blood cells of individuals that do not have PAH.

In one aspect of this embodiment, each polynucleotide is complementary to an RNA transcript, or a polynucleotide derived therefrom, of a gene comprising, or expressing a transcript comprising, a nucleic acid sequence selected from the group consisting of SEQ ID NOs:1-128. In another aspect of this embodiment, the plurality of polynucleotides comprises polynucleotides that are complementary to an RNA transcript, or a nucleotide derived therefrom, of at least two genes comprising, or expressing a transcript comprising, a nucleic acid sequence selected from the group consisting of SEQ ID NOs:1-128. In yet another aspect of this embodiment, the plurality of polynucleotides comprises polynucleotides that are complementary to an RNA transcript, or a nucleotide derived therefrom, of at least five genes, at least 10 genes, at least 25 genes, at least 50 genes, at least 100 genes, or up to all of the genes, comprising, or expressing a transcript comprising, a nucleic acid sequence selected from the group consisting of SEQ ID NOs:1-128. In one aspect, the polynucleotide probes are immobilized on a substrate. In another aspect, the polynucleotide probes are hybridizable array elements in a microarray. In yet another aspect, the polynucleotide probes are conjugated to detectable markers.

Yet another embodiment of the invention relates to a method to monitor the treatment of a patient with pulmonary arterial hypertension (PAH), comprising: (a) detecting the level of expression of at least one gene in a sample of peripheral blood cells isolated from a patient undergoing treatment for PAH, wherein the gene is chosen from a gene comprising, or expressing a transcript comprising, a nucleic acid sequence selected from the group consisting of SEQ ID NOs:1-101; and (b) comparing the level of expression of comparing the level of expression of the gene or genes detected in the patient sample to the level of expression of the gene or genes in a prior sample of peripheral blood cells from the patient and to a level of expression of the gene or genes in peripheral blood cells from normal controls that do not have PAH, wherein detection of a change in the level of expression of the gene or genes, as compared to the level of expression in the prior sample, toward the level of the expression of the gene in a normal control sample, indicates that the treatment for pulmonary hypertension is producing a beneficial result.

Another embodiment of the present invention relates to a method to diagnose a pulmonary disease or condition in a patient, comprising: (a) detecting in a sample of peripheral blood cells from a patient to be tested the level of expression of at least one biomarker chosen from a panel of biomarkers whose expression in peripheral blood cells has been associated with a pulmonary disease as measured by either upregulation or downregulation of biomarker expression in peripheral blood cells from patients with the pulmonary disease as compared to the level of expression of the biomarkers in peripheral blood cells from normal controls that do not have the pulmonary disease; (b) comparing the level of expression of the biomarker or biomarkers detected in the patient sample to a level of expression of the biomarker or biomarkers that has been associated with the pulmonary disease and a level of expression of the biomarker or biomarkers that has been associated with normal controls; and (c) diagnosing the pulmonary disease in the patient if the expression level of the biomarker or biomarkers in the patient sample is statistically more similar to the expression level of the biomarker or biomarkers that has been associated with the pulmonary disease than the expression level of the biomarker or biomarkers that has been associated with the normal controls. In one aspect, the disease or condition is a heart disease.

Yet another embodiment of the present invention relates to a method to identify a compound with the potential to treat pulmonary arterial hypertension (PAH), comprising: (a) contacting a test compound with a cell that expresses a gene chosen from a gene comprising, or expressing a transcript comprising, a nucleic acid sequence selected from the group consisting of SEQ ID NOs:1-128; and (b) identifying compounds that: (i) increase the expression or activity of the gene or protein encoded thereby if the expression of the gene is downregulated in peripheral blood cells of patients with pulmonary arterial hypertension as compared to the expression or activity of the gene or encoded protein in peripheral blood cells of normal controls; or (ii) decrease the expression or activity of the gene or protein encoded thereby if the expression of the gene is upregulated in peripheral blood cells of patients with pulmonary arterial hypertension as compared to the expression or activity of the gene or encoded protein in peripheral blood cells of normal controls. In one aspect of this embodiment, the cell expresses a nucleic acid molecule (represented by SEQ ID NO:94) encoding adrenomedullin, and step (b) comprises identifying compounds that decrease the expression or activity of adrenomedullin or the gene encoding adrenomedullin. In another aspect of this embodiment, the cell expresses a nucleic acid molecule (represented by SEQ ID NO:91) encoding endothelial cell growth factor-1, and step (b) comprises identifying compounds that decrease the expression or activity of endothelial cell growth factor-1 or the gene encoding endothelial cell growth factor-1.

### BRIEF DESCRIPTION OF THE FIGURES OF THE INVENTION

FIGS. 1A and 1B are dendrograms of PAH and Non-PAH samples, clustered using centered correlation and average linkage. FIG. 1A shows unsupervised clustering based on 2906 genes; FIG. 1B shows supervised clustering based on 106 genes.

FIGS. 2A and 2B show quantitative PCR measurements of gene expression of endothelial cell growth factor-1 (ECGF-1) from PBMC samples of patients with PAH and normal volunteers; FIG. 2A represents gene expression of ECGF-1 in patients with PAH compared to normal volunteers from the microarray cohort, and FIG. 2B represents ECGF-1 expression in patients with PAH compared to normal volunteers from the prospective cohort.

FIGS. 3A and 3B show quantitative PCR measurements of gene expression of adrenomedullin (ADM) from PBMC samples of patients with PAH; FIG. 3A represents gene expression of ADM in patients with PAH compared to normal volunteers from the microarray cohort, and FIG. 3B represents ADM expression in patients with PAH compared to normal volunteers from the prospective cohort.

FIG. 4 shows quantitative PCR measurements of gene expression of Herpesvirus entry mediator (HVEM) from PBMC samples of patients with IPAH and s-PAH measured in the prospective cohort.

### DETAILED DESCRIPTION OF THE INVENTION

The present invention generally relates to the identification of a large number of genes that are regulated differentially in different forms of pulmonary arterial hypertension (PAH), and particularly, to the identification of how these genes are regulated during disease. In addition, this invention generally relates to diagnostic and prognostic assays and kits for severe pulmonary arterial hypertension and other lung disorders. More specifically, as discussed above, pulmonary arterial hypertension (PAH) is associated with an altered cytokine, chemokine and growth factor milieu of circulating cells (19; 22; 31; 32). These changes, combined with the genetic susceptibility to the development of PAH and the possibility of autoimmune and inflammatory etiologies of the disease, support the present inventors' hypothesis that peripheral blood mononuclear cells (PBMCs) from patients with PAH would have an altered gene expression pattern compared to normal individuals. The present inventors also hypothesized that this PBMC expression pattern may carry disease specific information compared to the PBMC expression of normal individuals. This approach has previously been utilized in the study of autoimmune diseases such as multiple sclerosis, systemic lupus erythematosus (SLE), rheumatoid arthritis (RA) and psoriatic arthritis, as well as kidney disease such as IgA nephropathy and lupus nephritis (33-36). However, to the present inventors' knowledge, the approach has not been used in pulmonary disorders prior to the present invention. Recent work has demonstrated that the gene expression profile of PBMCs compared between normal individuals is remarkably homogeneous, and distinct from the expression profiles of diseased individuals (37). Here the inventors have used PBMCs as a surrogate tissue to distinguish patients with PAH from normal individuals by gene expression profiling. The present inventors demonstrate, for the first time, that global expression profiling of peripheral blood cells aids in the diagnosis of a chronic pulmonary disease.

Methodologies for large scale molecular profiling of diseased tissues are well established with proven efficacy both diagnostically and prognostically (39-42). Recently, microarray analysis of surrogate tissues has been used to gain disease specific information. A number of investigators have employed this technology to document differences in gene expression of peripheral blood cells in a number of disease states (33, 35, 43, 44). The present inventors are believed to be the first to define gene expression of PBMCs to explore the diagnosis of PAH.

However, it is not intuitive that circulating blood cells will carry information related to pulmonary hypertension. T lymphocytes, which recognize antigens presented by PBMCs, respond to specific antigens, but not with a disease-specific response to a single antigen. Therefore, while screening PBMCs might be useful to distinguish between normal patients and those with an autoimmune disease due to exposure to inflammatory conditions or infection, the application of such technology to distinguish between forms of a specific disease such as PAH is not intuitively operable. More particularly, because the pulmonary artery pressure is very similar in patients with primary and secondary pulmonary arterial hypertension, as is the degree of vessel remodeling and the histology of the vascular lesions, it was not predictable that peripheral blood cells would be able to distinguish between the diseases. Indeed, it is not possible to differentiate between severe forms of pulmonary hypertension based on histology. However, the present inventors have successfully applied PBMC gene expression profiling to the study of pulmonary arterial hypertension and have unexpectedly uncovered a gene expression profile which effectively distinguishes between patients with idiopathic pulmonary arterial hypertension and patients with secondary forms of pulmonary hypertension. Given that such discrimination is not possible using histological analysis, this discovery is both surprising and represents a significant advance in the field. The idea that circulating blood cells may carry highly disease-specific information (in contrast to information that is consistent with some inflammatory process, as in autoimmune disease or infection) is believed to be completely novel and can be applied to other diseases, including other lung diseases.

According to the present invention, the terms “pulmonary arterial hypertension” (PAH) and “pulmonary hypertension” (PH) can be used interchangeably to describe the condition characterized by pressure elevation in the pre-capillary pulmonary vasculature of the lung. Primary pulmonary hypertension (PPH) is one type of disease within the spectrum of diseases characterized by pulmonary arterial hypertension, also referred to herein as idiopathic pulmonary arterial hypertension (IPAH), and secondary pulmonary hypertension (SPH) is another, also referred to herein as pulmonary arterial hypertension related to a secondary cause (s-PAH).

Currently, IPAH is a diagnosis of exclusion. Because of the rarity of this disease and the difficulty inherent in making this diagnosis, the average time from onset of symptoms to appropriate diagnosis of IPAH is 2 years. The ability to aid in the diagnosis of IPAH with a blood test would be of significant benefit in its management. Such a test would facilitate the earlier diagnosis of this disease, when treatment might be more effective. It would also decrease the need for a number of expensive and invasive studies used to exclude secondary causes of pulmonary arterial hypertension. The present invention can also be used to aid in better defining certain phenotypes of this disease. Moreover, the present invention can be used to monitor progression of a disease and/or the efficacy of disease treatments.

In addition, the present inventors have uncovered a number of genes not previously recognized to play a role in the disease process of pulmonary hypertension, which can now be studied in more detail and/or be used as targets for the discovery of other modulators of disease or therapeutic agents.

Pulmonary arterial hypertension constitutes a wide spectrum of diseases that result in similar histopathologic and clinical phenotypes. Although it is increasingly accepted that patients who develop PAH have a genetic predisposition, the exact nature of these genetic anomalies and how the genotype interacts with various environmental factors remains unclear (45-48). Circulating blood cells may carry disease specific information either because of inherent genomic alterations or because of alterations in their local environment. Furthermore, the gene expression of PBMCs may provide disease specific information due to inflammatory and autoimmune mechanisms that likely play important roles in the development of PAH (7, 10, 11, 17, 23, 24, 49). The present inventors have identified a gene expression pattern that accurately distinguishes patients with PAH from individuals without PAH. The present inventors have also identified a number of genes which may be associated with the pathobiology of pulmonary arterial hypertension. Lastly, the present inventors have prospectively tested whether genes initially identified by microarray analysis could discriminate patients with idiopathic pulmonary arterial hypertension (IPAH) from those with secondary pulmonary arterial hypertension (S-PAH) and normal controls using quantitative PCR.

Several features of the study disclosed herein make it unique and important. This study was designed to emphasize the importance of measure validation. Due to the high measurement-to-sample ratio inherent in microarray studies, steps must be taken to avoid “over-fitting” any predictive model (50). For this reason, the inventors employed both independent and “leave-one-out” cross-validation of their predictor. In the studies described in the Examples below, this was accomplished using a training set of two-thirds of the total number of arrays. The remaining one-third of the samples was used as a test set to determine, in a blinded fashion, the predicted value of the class prediction analysis. The blinded, prospectively analyzed, one-third contingent was accurately classified in all cases. This independent blinded data set represents the most stringent manner of analyzing the quality of a prediction rule.

In the absence of such an independent data set, two commonly used procedures are suited to access the quality of a prediction rule, 1) cross validation and 2) permutation testing. Both of the procedures were employed in the experiments described herein. The “leave-one-out” cross-validation class prediction framework provides cross validation of prediction results by leaving out a portion of the data, building the prediction rule on the remaining data, and predicting the labels of the left out data. The sample size for the present inventors' data set was appropriately suited for such an analysis. In this manner, all specimens could be accurately classified using 3 of 5 classification algorithms. The remaining 2 algorithms resulted in a single miss-classification each.

Cross validation using these class prediction models is an important aspect of the class prediction process but is not sufficient for accessing the significance of a classification result. If there were to exist small cross validation error rates, this would not guarantee that the sub-classification of specimens would actually be correct. In the present inventors' schema, permutation testing was used to access the significance of the cross validation error rate. This procedure was performed by randomly permuting class labels among the gene expression measurements (for example normal vs. pulmonary hypertension) considering 2000 permutations of the class labels. The proportion of the permuted data sets that have a classification error rate less than or equal to the unpermuted misclassification rate serve as the achieved significance level in a test against the null hypothesis—that there is no difference in gene expression profiles between the two classes.

The genes and gene ontology (GO) categories identified as being differentially expressed in PAH vs. normal controls (see Examples) are of significant biologic interest. The inventors selected 2 genes from the 101 gene list that distinguishes PAH from normal individuals and 1 gene from the 28 gene list that was differentially expressed between patients with IPHA and s-PAH. These genes were selected both to confirm the results of the microarray prospectively, but also because of their perceived biologic interest. Adrenomedullin (represented herein by SEQ ID NO:94; increased in PAH vs. normal) is a potent pulmonary vascular vasodilator (59). Plasma levels of adrenomedullin are elevated in idiopathic and secondary forms of pulmonary hypertension and its use as a therapeutic inhalational agent in the treatment of PAH is currently under investigation (58, 60, 61). Endothelial cell growth factor-1 (represented herein by SEQ ID NO:91; increased in PAH vs. normal) is a potent angiogenic factor and is increased in expression in a number of malignancies (62, 63, 64). As the plexiform lesion of PAH is composed of an abnormal proliferating endothelial cell phenotype, an association between patients with PAH and increased expression of ECGF-1 is of significant interest. Furthermore, the differential expression of HVEM (represented herein by SEQ ID NO:102) between patients with IPAH and s-PAH (increased in s-PAH vs. IPAH) is potentially relevant in the context of the inventors' recent reports of an association between IPAH and HHV-8 infection (23).

The present inventors' work represents a novel approach to the identification and classification of pulmonary arterial hypertension. Lack of access to the site of pathology, i.e., the lung vessels, has severely limited the study of PAH disease progression at both histologic and molecular levels. The ability to distinguish patients with PAH by examining peripheral blood has significant implications for both diagnosis and screening for this disease. Differences in gene expression may also provide insight into the pathobiology of PAH and may be promising as a means to monitor the effects of drug treatment.

Of the more than 2900 human genes screened, the present inventors have identified multiple genes, the expression of which is regulated differentially in peripheral blood cells, or PBC (also referred to herein as peripheral blood mononuclear cells, or PBMC) of patients with pulmonary hypertension as compared to subjects without pulmonary hypertension. Expression of the genes can be further categorized based on the regulation of expression of the genes in PBCs of patients with idiopathic pulmonary arterial hypertension (IPAH) versus secondary pulmonary arterial hypertension (s-PAH) versus normal controls. More particularly, the genes can be grouped into the following main categories: (1) genes that are selectively (i.e., exclusively or uniquely) upregulated in PBCs of patients with pulmonary arterial hypertension as compared to normal controls (Table 3); and (2) genes that are selectively downregulated in PBCs of patients with pulmonary arterial hypertension as compared to normal controls (Table 3).

Table 3 shows the identity of 101 genes that were identified by the present inventors to be significantly regulated (up or down) in patients with PAH as compared to normal controls, sorted in ascending order of the p-value (at p<0.001) of the univariate test (i.e., decreasing statistical significance as one moves down the table). The geometric means of intensities and the fold change are shown for the expression of each transcript in peripheral blood cells from normal controls as compared to patients with PAH. Using this information, one can clearly see whether a given gene is upregulated or downregulated in the peripheral blood cells of patients with PAH as compared to the normal control.

Table 4 of the invention shows a partial list of genes from Table 3 with significant differences in the expression between patients diagnosed with PAH and normal controls. These are genes that are deemed by the present inventors to be of high potential biologic interest, although any of the genes described herein are useful in the present invention.

Table 5 of the invention shows a list of 28 genes found to be differentially expressed between patients with IPAH versus s-PAH by supervised class comparison. These genes are significantly different at the α=0.01 level. The fold change in this table is expressed as IPAH/s-PAH. Note that there is one biomarker in common between Tables 3 and 5 (SEQ ID NO:84; probe set ID number U80184_ma1_at; Homo sapiens FLII gene).

Table 6 of the invention shows Gene Ontology (GO) categories with an abundance of differentially expressed genes in PAH vs. normal individuals. Gene symbol and Gene Bank ID are listed and the sequence represented by each is incorporated herein by reference in its entirety. The zscore assigned to each GO category reflects the degree to which the expression of genes within the category was greater then expected by chance alone. Fold change indicates PAH in relation to normal (PAH/Normal).

The nucleotide sequences representing the biomarkers shown in Tables 3-5 are represented herein by SEQ ID NOs:1-128. The nucleic acid sequences represented by SEQ ID NOs:1-128 include transcripts or nucleotides derived therefrom (e.g., cDNA) expressed by the gene biomarkers referenced in Tables 3, 4 and 5. It is to be understood that the present invention expressly covers additional genes that can be elucidated using substantially the same techniques used to identify the genes in Tables 3, 4 and 5 and that any of such additional genes can be used in the methods and products described herein for the genes and probe sets in Tables 3, 4 and 5. Any reference to database Accession numbers or other information regarding the genes and probe sets in any of Tables 3, 4, 5 or 6 is hereby incorporated by reference in its entirety. For each biomarker listed in Tables 3 and 5, the following information is provided: (1) the probe set ID number given by Affymetrix™ for the set of features on the array representing the indicated gene; (2) the parametric p-value, indicating the statistical significance of that individual gene expression difference; (3) the mean intensity of expression of each gene in normal (non-PAH) individuals and in PAH patients (Table 3) or in IPAH patients and s-PAH patients (Table 5); (4) the fold-change in geometric intensities of normal/PAH (Table 3) or IPAH/s-PAH (Table 5); (5) the HUGO-approved symbol for the gene, where one exists; (6) the sequence identifier representing a nucleotide sequence found in or transcribed by the gene; and (7) the name or title of the gene, where one is given. It is noted that in the event that two probe sets in any of the tables appear to refer to a single gene, such duplications have been maintained because they are believed to reflect different splice variants of that gene. In such a case, the associated sequence files will reflect the different splicotypes for that gene.

In addition, the present invention will also be useful for the validation in other studies of the clinical significance of many of the specific biomarkers described herein, as well as the identification of preferred biomarker profiles, highly sensitive biomarkers, and targets for the design of novel therapeutic products and strategies.

Accordingly, in one embodiment of the present invention, the genes identified as being regulated in PBCs of patients with pulmonary hypertension can be used as endpoints or markers (also called “biomarkers”) in a diagnostic or prognostic assay for PAH. The biomarkers useful in the present invention may include any of the genes listed in any of the tables presented herein (e.g., Tables 3-6), with the genes listed in Tables 3-5 being preferred. In a preferred embodiment, the biomarkers useful in the present invention correspond to a gene comprising, or expressing a transcript comprising, a nucleic acid sequence selected from any of SEQ ID NOs:1-128. Diagnostic assays include assays that determine whether a patient has overt PAH or preclinical stage PAH, and can include a more specific diagnosis of IPAH or s-PAH. Prognostic assays can be used to stage a patient's development of PAH, predict a patient's outcome or disease progression, and/or monitor the effectiveness of various treatment protocols on PAH.

The term “biomarker” as used herein can refer to an endpoint gene described herein or to the protein encoded by that gene. In addition, the term “biomarker” can be generally used to refer to any portion of such a gene or protein that can identify or correlate with the full-length gene or protein, for example, in an assay of the invention. According to the present invention, an “endpoint gene” or “biomarker gene” is any gene, the expression of which is regulated (up or down) in a patient with a condition as compared to a normal control. Selected sets of one, two, three, and more preferably several more of the genes of this invention (up to the number equivalent to all of the genes, including any intervening number, in whole number increments, e.g., 1, 2, 3, 4, 5, 6 . . . ) can be used as end-points for rapid diagnostics or prognostics for PAH. Preferably, larger numbers of the genes identified in any one or more of Tables 3-6 are used in an assay of the invention (e.g., at least 10 genes or more), since the accuracy of the assay improves as the number of genes screened increases.

According to the present invention, the method includes the step of detecting the expression of at least one, and preferably more than one (e.g., 2, 3, 4, 5, 6, . . . and so on, in increments of whole numbers up to all of the genes) of the genes that have now been shown to be selectively regulated in PBMCs of patients with PAH by the present inventors. As used herein, the term “expression”, when used in connection with detecting the expression of a gene of the present invention, can refer to detecting transcription of the gene and/or to detecting translation of the gene. To detect expression of a gene refers to the act of actively determining whether a gene is expressed or not. This can include determining whether the gene expression is upregulated as compared to a control, downregulated as compared to a control, or substantially unchanged as compared to a control. Therefore, the step of detecting expression does not require that expression of the gene actually is upregulated or downregulated, but rather, can also include detecting no expression of the gene or detecting that the expression of the gene has not changed or is not different (i.e., detecting no significant expression of the gene or no significant change in expression of the gene as compared to a control).

The present method includes the step of detecting the expression of at least one gene that is selectively regulated in PBCs of a patient with PAH (any form). In a preferred embodiment, the step of detecting includes detecting the expression of at least 2 genes, and preferably at least 3 genes, and more preferably at least 4 genes, and more preferably at least genes, and more preferably at least 6 genes, and more preferably at least 7 genes, and more preferably at least 8 genes, and more preferably at least 9 genes, and more preferably at least 10 genes, and more preferably at least 11 genes, and more preferably at least 12 genes, and more preferably at least 13 genes, and more preferably at least 14 genes, and more preferably at least 15 genes, and more preferably at least 20 genes, and more preferably at least 25 genes, and more preferably at least 50 genes, and more preferably at least 75 genes, and more preferably at least 100 genes, and so on, in whole integer increments (i.e., 1, 2, 3, . . . 10, 11, 12, . . . 35, 36, 37, . . . 56, 57, 58, . . . 98, 99, 100, . . . 128), up to detecting expression of all of the genes that can be used to detect PAH as disclosed herein. Analysis of a number of genes greater than one can be accomplished simultaneously, sequentially, or cumulatively. As discussed above, it is preferred that several to most of the genes be detected in the present methods, as the accuracy of the method improves as the number of genes detected increases. However, it is to be understood that in some circumstances, it may be desirable and sufficient to detect the expression of only one or a few genes.

In the diagnostic or prognostic method of the present invention, the gene(s) to be detected are preferably selected from the genes described in any one or more of Tables 3, 4 or 5, or any combination thereof, and particularly include a gene comprising, or expressing a transcript comprising, a nucleic acid sequence selected from the group consisting of SEQ ID NOs:1-128. These tables and genes have been discussed above in detail and disclose genes that the present inventors have discovered to be selectively regulated in the PBCs of patients with pulmonary hypertension and particularly, in patients with primary and/or secondary pulmonary arterial hypertension. More specifically, these tables disclose the manner in which the genes are regulated (e.g., upregulated or downregulated) in a patient with PAH as compared to a normal control. In addition, by comparing the patients with primary versus secondary pulmonary arterial hypertension, one can also see that at least some of the genes are useful as markers to distinguish between these diseases (e.g., see Table 5).

It is to be understood that the organization of various genes into the present tables is for purposes of illustrating various experimental data described in the Examples section. The selection of genes to be detected in any given method can include any one or more of the genes in any of the Tables 3-6, and preferably Tables 3-5, and can include the detection of any combination of two or more of the genes in of these Tables, and preferably includes the detection of any combination of multiple genes in any of these Tables, including detection of a gene comprising, or expressing a transcript comprising, a nucleic acid sequence selected from any one or more of SEQ ID NOs:1-128. It is not mandatory that a given assay be restricted to the detection of all of the various genes in a single table, or to at least one gene in each table. In addition, one may choose also to detect other genes that are believed to be useful in the evaluation of a patient for PAH, and therefore, the present method is not limited exclusively to detection of the genes identified herein, although the invention is primarily directed to the detection of one or more of these genes and includes the detection of at least one or more of these genes. In addition, provided with this disclosure, one of skill in the art may proceed to identify additional genes that are differentially regulated in the PBCs of patients with PAH, and detection of any of such genes may be used in the methods of the present invention, including in combination with detection of any of the genes disclosed herein. Indeed, the present inventors have now provided a powerful method to detect and evaluate biomarkers for PAH and have also provided data demonstrating the application of such technology.

Given the knowledge of the genes regulated in PAH according to the present invention, one of skill in the art will be able to select one or more genes to detect in a method of the present invention, and the selection of the one or more genes can be determined by different factors. For example, certain subsets of the genes are useful for detecting patients that have pulmonary hypertension, regardless of the form of PAH (e.g., Tables 3 and 4, and SEQ ID NOs:1-101). Other subsets of genes are useful for detecting patients that have primarily PAH (IPAH) versus patients that have secondary PAH (S-PAH) (e.g., see Table 5 and SEQ ID NOs:84 and 102-128).

In one aspect, it may be desirable to preferentially select those genes for detection that are particularly highly regulated in patients with PAH (either form) in that they display the largest increases or decreases in expression levels in patients as compared to normal controls or as compared to the other form of PAH. The detection of such genes can be advantageous because the endpoint may be more clear and require less quantitation. The relative expression levels of the genes identified in the present invention are listed in the tables.

According to the present invention, a “baseline” or “control” can include a normal or negative control and/or a disease or positive control, against which a test level of gene expression can be compared. Therefore, it can be determined, based on the control or baseline level of gene expression, whether a sample to be evaluated for PAH has a measurable difference or substantially no difference in gene expression, as compared to the baseline level. In one aspect, the baseline control is a indicative of the level of gene expression as expected in the PBCs of a normal (e.g., healthy, negative control, non-PH) patient. Therefore, the term “negative control” used in reference to a baseline level of gene expression typically refers to a baseline level of expression from a population of individuals which is believed to be normal (i.e., not having or developing PAH). In some embodiments of the invention, it may also be useful to compare the gene expression in a test sample of PBCs to a baseline that has previously been established from a patient or population of patients with PAH. Such a baseline level, also referred to herein as a “positive control”, refers to a level of gene expression established in PBCs from one or preferably a population of individuals who had been positively diagnosed with PAH.

In one embodiment, when the goal is to monitor the progression or regression of PAH in a patient, for example, to monitor the efficacy of treatment of the disease or to determine whether a patient that appears to be predisposed to the disease begins to develop the disease, one baseline control can include the measurements of gene expression in a sample of PBCs from the patient that was taken from a prior test in the same patient. In this embodiment, a new sample is evaluated periodically (e.g., at annual or more regular physicals), and any changes in gene expression in the patient PBCs as compared to the prior measurement and most typically, also with reference to the above-described normal and/or positive controls, are monitored. Monitoring of a patient's PBC gene expression profile tumor can be used by the clinician to prescribe or modify treatment for the patient based on whether any differences in gene expression in the PBCs is indicated.

In a preferred embodiment, the control or baseline levels of gene expression are obtained from PBCs collected from “matched individuals”. According to the present invention, the phrase “matched individuals” refers to a matching of the control individuals on the basis of one or more characteristics, such as gender, age, race, or any relevant biological or sociological factor that may affect the baseline of the control individuals and the patient (e.g., preexisting conditions, consumption of particular substances, levels of other biological or physiological factors). The number of matched individuals from whom control samples must be obtained to establish a suitable control level (e.g., a population) can be determined by those of skill in the art, but should be statistically appropriate to establish a suitable baseline for comparison with the patient to be evaluated (i.e., the test patient). The values obtained from the control samples are statistically processed using any suitable method of statistical analysis to establish a suitable baseline level using methods standard in the art for establishing such values. It will be appreciated by those of skill in the art that a baseline need not be established for each assay as the assay is performed but rather, a baseline can be established by referring to a form of stored information regarding a previously determined control level of gene expression. Such a form of stored information can include, for example, but is not limited to, a reference chart, listing or electronic file of population or individual data regarding “normal” (negative control) or PAH-positive gene expression; a medical chart for the patient recording data from previous evaluations; or any other source of data regarding control gene expression that is useful for the patient to be diagnosed or evaluated.

Expression of the transcripts and/or proteins encoded by the genes of the invention is measured by any of a variety of known methods in the art. In general, the nucleic acid sequence of a nucleic acid molecule (e.g., DNA or RNA) in a patient sample can be detected by any suitable method or technique of measuring or detecting gene sequence or expression. Such methods include, but are not limited to, polymerase chain reaction (PCR), reverse transcriptase-PCR (RT-PCR), in situ PCR, quantitative PCR (q-PCR), in situ hybridization, Southern blot, Northern blot, sequence analysis, microarray analysis, detection of a reporter gene, or other DNA/RNA hybridization platforms. For RNA expression, preferred methods include, but are not limited to: extraction of cellular mRNA and Northern blotting using labeled probes that hybridize to transcripts encoding all or part of one or more of the genes of this invention; amplification of mRNA expressed from one or more of the genes of this invention using gene-specific primers, polymerase chain reaction (PCR), quantitative PCR (q-PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene. The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

Methods to measure protein expression levels of selected genes of this invention, include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry, and assays based on a property of the protein including but not limited to DNA binding, ligand binding, or interaction with other protein partners.

Nucleic acid arrays are particularly useful for detecting the expression of the genes of the present invention. The production and application of high-density arrays in gene expression monitoring have been disclosed previously in, for example, PCT Publication No. WO 97/10365; PCT Publication No. WO 92/10588; U.S. Pat. No. 6,040,138; U.S. Pat. No. 5,445,934; or PCT Publication No. WO 95/35505, all of which are incorporated herein by reference in their entireties. Also for examples of arrays, see Hacia et al. (1996) Nature Genetics 14:441-447; Lockhart et al. (1996) Nature Biotechnol. 14:1675-1680; and De Risi et al. (1996) Nature Genetics 14:457-460, each of which is incorporated by reference in its entirety. In general, in an array, an oligonucleotide, a cDNA, or genomic DNA, that is a portion of a known gene, occupies a known location on a substrate. A nucleic acid target sample is hybridized with an array of such oligonucleotides and then the amount of target nucleic acids hybridized to each probe in the array is quantified. One preferred quantifying method is to use confocal microscope and fluorescent labels. The Affymetrix GeneChip™ Array system (Affymetrix, Santa Clara, Calif.) and the Atlas™ Human cDNA Expression Array system are particularly suitable for quantifying the hybridization; however, it will be apparent to those of skill in the art that any similar systems or other effectively equivalent detection methods can also be used. In a particularly preferred embodiment, one can use the knowledge of the genes described herein to design novel arrays of polynucleotides, cDNAs or genomic DNAs for screening methods described herein. Such novel pluralities of polynucleotides are contemplated to be a part of the present invention and are described in detail below.

Suitable nucleic acid samples for screening on an array contain transcripts of interest or nucleic acids derived from the transcripts of interest (i.e., transcripts derived from the genes associated with PAH of the present invention). As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like. Preferably, such a sample is a total RNA preparation of a biological sample (e.g., peripheral blood mononuclear cells or PBMCs). More preferably in some embodiments, such a nucleic acid sample is the total mRNA isolated from such a biological sample. Preferably, the nucleic acids for screening are obtained from a homogenate of cells (e.g., peripheral blood mononuclear cells or PBMCs).

In general, typical clinical samples include, but are not limited to, sputum, blood, blood cells (e.g., peripheral blood mononuclear cells), tissue or fine needle biopsy samples, urine, peritoneal fluid, and pleural fluid, or cells therefrom. The present invention is primarily related to the detection of genes in peripheral blood mononuclear cells (PBMC, which can also be abbreviated as PBC).

In one embodiment, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids to achieve quantitative amplification. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high-density array may then include probes specific to the internal standard for quantification of the amplified nucleic acid. Other suitable amplification methods include, but are not limited to polymerase chain reaction (PCR) Innis, et al., PCR Protocols. A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al, Proc. Nat. Acad. Sci. USA, 87: 1874 (1990)).

Nucleic acid hybridization involves contacting a probe and target nucleic acid under conditions where the probe and its complementary target can form stable hybrid duplexes through complementary base pairing. As used herein, hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety. Nucleic acids that do not form hybrid duplexes are washed away from the hybridized nucleic acids and the hybridized nucleic acids can then be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC). Other hybridization conditions, and for example, those most useful with nucleic acid arrays, will be known to those of skill in the art.

The hybridized nucleic acids are detected by detecting one or more labels attached to the sample nucleic acids. The labels may be incorporated by any of a number of means well known to those of skill in the art. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, yellow fluorescent protein and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The method of the present invention includes a step of comparing the results of detecting the expression of the one or more genes that are selectively regulated in patients with PAH as compared to a control (baseline, normal control or patient with an alternate form of PAH) in order to determine whether there is any observed change or difference in expression of each gene in the patient as compared to the control. As discussed above, the present inventors have identified the expression profile of multiple genes that are differentially regulated in PBCs of patients with IPAH and s-PAH, as compared to each other and as compared to a “normal” control (i.e., a patient that does not have or can not be detected to have PAH), including the manner in which the genes are regulated (i.e., up- or downregulated). Therefore, one can determine whether peripheral blood cells from a test patient have a gene expression profile that is statistically substantially similar to the profile of gene expression of a patient with PAH, and particularly, with IPAH or s-PAH, or whether a profile of gene expression in the peripheral blood cells of the test patient is statistically more similar to the negative or normal, non-disease control.

According to the present invention, an expression profile is substantially similar to a given profile of expression established for a group (e.g., PAH group, IPAH group, s-PAH group, normal control group) if the expression profile of the gene or genes detected (including the identity of the gene, the manner in which expression is regulated, and/or the level of expression of the gene) is similar enough to the expected result so as to be statistically significant (i.e., with at least a 95% confidence level, or p<0.05, and more preferably, with a confidence level of p<0.01, and even more preferably, with a confidence level of p<0.005, and even more preferably, with a confidence level of p<0.001). Software programs are available in the art that are capable of analyzing the expression of multiple genes and determining whether differences from a control are significant or not significant. For example, as discussed in the Examples, the gene expression measurements determined in patient samples were mean-centered and analyzed using the clustering, class comparison and class discovery functions of BRB ArrayTools and genes were selected that met the p value requirement (0.001). In addition, statistical analysis methods are known in the art and described herein (see above and the Examples) that are preferably used to analyze the expression data generated for patient samples (e.g., independent and “leave-one-out” cross-validation and/or permutation testing).

By way of example, detection of the regulation of the expression of a gene in the “manner” associated with the established group, at a minimum, refers to the detection of the regulation of a gene that has now been shown by the present inventors to be selectively regulated in PBCs of patients having PAH, in the same direction (i.e., upregulation or downregulation) and at a similar or comparable level, as compared to a normal or baseline control established for the expression of that gene. Preferably, a gene identified as being upregulated or downregulated, as compared to a baseline control, is regulated in the same direction as the level of expression of the gene that is seen in established or confirmed patients with PAH as compared to a normal control. In other words, if “gene X” is upregulated in patients with PAH as compared to a normal control based on the inventors' discovery presented herein, then one determines whether the expression of gene X is upregulated in a patient test sample as compared to a normal control, or whether the expression of gene X is more similar to the level of expression of the normal control. In one aspect of the invention, a gene identified as being upregulated or downregulated as compared to a baseline control according to the invention is regulated in the same direction and to at least about 10%, and more preferably at least 20%, and more preferably at least 25%, and more preferably at least 30%, and more preferably at least 35%, and more preferably at least 40%, and more preferably at least 45%, and more preferably at least 50%, and preferably at least 55%, and more preferably at least 60%, and more preferably at least 65%, and more preferably at least 70%, and more preferably at least 75%, and more preferably at least 80%, and more preferably at least 85%, and more preferably at least 90%, and more preferably at least 95%, or even higher (e.g., above 100%) of the level of expression of the gene that is seen in established or confirmed patients with PAH. Statistical significance should be at least p<0.05, and more preferably, at least p<0.01, and more preferably, p<0.005, and even more preferably, p<0.001. As discussed above, one of skill in the art can use software programs available in the art which use algorithms to analyze gene expression profiles and identify significant differences among samples and controls. In addition, one of skill in the art can apply various types of analyses as discussed above (e.g., cross-validation and/or permutation testing) to validate the results of the methods described herein.

A profile of individual gene markers to use in a method of the invention, including a matrix of two or more markers, can be generated by one or more of the methods described above. According to the present invention, a profile of the genes regulated in a PBC sample refers to a reporting of the expression level of a given gene from any one or more of the tables presented herein, which, based on the knowledge of the regulation of the genes provided by the tables, includes a classification of the gene with regard to how the gene is regulated in PBCs of a patient with pulmonary arterial hypertension, and may include a classification of how the gene is regulated in patients with idiopathic pulmonary arterial hypertension versus secondary pulmonary arterial hypertension. For example, if a specific gene is identified as being expressed by a peripheral blood cell sample in a test patient, the profile for the blood cell sample will include the reporting of the expression of this gene as compared to one or more baseline controls (e.g., a negative/normal and/or a positive/PH control). Preferably, the profile includes data for more than one (e.g., at least two), and preferably several genes (e.g., at least five, six, seven, eight, nine, ten, or more genes), such that a profile for the patient sample is created that can be compared to the control(s). The data can be reported as raw data, and/or statistically analyzed by any of a variety of methods, and/or combined with any other prognostic marker(s) for pulmonary arterial hypertension, including any markers that are expressed in cells or tissues other than PBCs and are useful for evaluating PAH in a patient. Prior to the present invention, one of skill in the art would not have known to screen patient peripheral blood cells for the particular genes in the tables provided herein, and particularly for any combinations of these genes, and one of skill in the art would not have been able to classify these genes or combinations thereof on the basis of pulmonary arterial hypertension versus normal, or on the basis of one form of pulmonary hypertension versus another.

It will be appreciated by those of skill in the art that differences between the expression of genes in PBCs of patients with PAH and without PAH may be small or large. Some small differences may be very reproducible and therefore are preferred for use in the diagnostic and prognostic methods of the invention. For other purposes, large differences may be desirable for ease of detection of the regulatory activity. It will therefore be appreciated that the exact boundary between a positive diagnosis and a negative diagnosis can shift, depending on the goal of the screening assay, the patient samples, the number of genes to be screened and the baseline controls used. For some assays, a given patient may be sampled over time to detect the efficacy of a treatment, and so changes in gene expression from a disease state toward a normal state may be detected. In this case, the patient may still be positive for a given form of PAH as compared to a normal, disease-free control, but may show a shift toward the normal control gene expression profile if treatment is successful. In addition, the technique being used for detection as well as on the number of genes which are being tested may impact how the assay is evaluated by those of skill in the art.

The profile of genes provided as a result of the screening of peripheral blood cells of a patient can be used by the patient or physician for decision-making regarding the usefulness of therapies for PAH in general. The profile can be used to estimate how the disease is likely to respond and progress in any individual patient. Clinical trials can be developed to correlate the relationship between IPAH and s-PAH regulated genes and the biological behavior of the diseased tissues, including in response to particular treatments for pulmonary hypertension.

In one aspect of this embodiment of the invention, the profiling of genes expressed by peripheral blood cells can be extended to other diseases, and particularly, to other pulmonary diseases wherein diagnosis or prognosis of disease is difficult due to access to diseased tissue or difficulty distinguishing between subtypes of the disease based on conventional assays (e.g., histology). For example, as discussed above, using the guidance provided herein, it is within the ability of those of skill in the art to perform a de novo screening assay for the identification of genes regulated in peripheral blood cells in patients having a different disease, and particularly, a pulmonary disease, and to develop gene expression profiles for use in diagnostic and/or prognostic screening for these diseases. Moreover, one of skill in the art can use the techniques described herein to screen other gene arrays, including arrays of expressed tag sequences, to discover additional novel, genes that are regulated in the peripheral blood cells of patients with PAH. The extension of the gene profiles within PAH and to other diseases will allow for the development of a variety of diagnostic assays in such diseases, as well as the identification of additional targets for therapeutic strategies. Such diseases can include, but are not limited, to any heart diseases, and in particular can include interstitial lung disease, diabetes, high blood pressure, heart failure and scleroderma.

It is to be understood that to perform the methods of the present invention, one of skill in the art can make use of any commercially available nucleotide or protein array, wherein hundreds or thousands of genes could be detected if desired. However, in the present method, one would use such an array to selectively screen only for the genes that are described as being useful for detection of PAH as disclosed herein, or to screen for such genes plus any other genes that are known to be useful as a predictor or analysis tool for PAH. In addition, the array can be designed to test for more than one disease condition in order to confirm or rule out other potential causes of a patient condition. For example, one may design an assay to screen for PAH as described herein, and also for a second pulmonary disease. In the specifically designed assays described herein, expression of non-informative genes can effectively be “ignored” or not screened. Alternatively, one of skill in the art can prepare nucleotide or protein arrays that are specifically designed to test for the expression of any combination of the genes of interest as described herein, alone or in combination with any other combination of genes that may be useful in evaluating a patient for PAH.

Another embodiment of the present invention relates to a plurality of polynucleotides for the detection of the expression of genes that are selectively regulated in peripheral blood cells of patients with PAH. The plurality of polynucleotides consists of, or consists essentially of, at least two polynucleotide probes that are complementary to RNA transcripts, or nucleotides derived therefrom, of at least one gene that has been identified herein as being selectively regulated in the peripheral blood cells of patients with PAH, and is therefore distinguished from previously known nucleic acid arrays and primer sets. The plurality of polynucleotides within the above-limitation includes at least two or more polynucleotide probes (e.g., at least 2, 3, 4, 5, 6, and so on, in whole integer increments, up to all of the possible probes) that are complementary to RNA transcripts, or nucleotides derived therefrom, of at least one gene, and preferably, at least 2 or more genes identified by the present inventors. Such genes are selected from any of the genes listed in the tables provided herein and can include any number of genes, in whole integers (e.g., 1, 2, 3, 4, . . . ). Multiple probes can also be used to detect the same gene or to detect different splice variants of the same gene. In one aspect, each of the polynucleotides in the plurality is at least 5 nucleotides in length. In one aspect, the plurality of polynucleotides consists of at least two polynucleotides, wherein each polynucleotide is at least 5 nucleotides in length, and wherein each polynucleotide is complementary to an RNA transcript, or nucleotide derived therefrom, of a gene comprising, or expressing a transcript comprising, a nucleic acid sequence selected from the group consisting of SEQ ID NOs:1-128. In another aspect, the plurality of polynucleotides comprises polynucleotides that are complementary to an RNA transcript, or a nucleotide derived therefrom, of at least two genes comprising, or expressing a transcript comprising, a nucleic acid sequence selected from the group consisting of SEQ ID NOs:1-128. In another aspect, the plurality of polynucleotides comprises polynucleotides that are complementary to an RNA transcript, or a nucleotide derived therefrom, of at least five genes, at least 10 genes, at least 25 genes, at least 50 genes, at least 100 genes, or up to all of the genes, comprising, or expressing a transcript comprising, a nucleic acid sequence selected from the group consisting of SEQ ID NOs:1-128.

In one embodiment, it is contemplated that additional genes that are not regulated in the peripheral blood cells of patients with pulmonary hypertension, or that are not presently known to be regulated in the peripheral blood cells of patients with pulmonary hypertension, can be added to the set of genes to be identified by the plurality of polynucleotides. Such genes would not be random genes, or large groups of unselected human genes, as are commercially available now, but rather, would be specifically selected to complement the sets genes identified by the present invention. For example, one of skill in the art may wish to add to the above-described plurality of polynucleotides one or more polynucleotides corresponding to (useful for identifying) genes that are of relevance because they are expressed by a particular tissue of interest (e.g., pulmonary tissue), are associated with the particular disease (PAH) but not necessarily with peripheral blood cells, or are associated with a particular cell, tissue or body function. The development of additional pluralities of polynucleotides (and antibodies, as disclosed below), which include both the above-described plurality and such additional selected polynucleotides, are explicitly contemplated by the present invention. In addition, using the techniques described herein, one of skill in the art may identify additional genes that are regulated in the peripheral blood cells of patients with PAH, and polynucleotides derived from such genes can be included in the plurality of polynucleotides described herein.

According to the present invention, a plurality of polynucleotides refers to at least 2, and more preferably at least 3, and more preferably at least 4, and more preferably at least 5, and more preferably at least 6, and more preferably at least 7, and more preferably at least 8, and more preferably at least 9, and more preferably at least 10, and so on, in increments of one, up to any suitable number of polynucleotides, including polynucleotides representing all of the genes described herein (e.g., 106), 500, 1000, 10⁴, 10⁵, or at least 10⁶ or more polynucleotides.

In accordance with the present invention, an isolated polynucleotide, or an isolated nucleic acid molecule, is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. The polynucleotides useful in the plurality of polynucleotides of the present invention are typically a portion of a gene (sense or non-sense strand) of the present invention that is suitable for use as a hybridization probe or PCR primer for the identification of a full-length gene (or portion thereof) in a given sample (e.g., a peripheral blood cell sample). An isolated nucleic acid molecule can include a gene or a portion of a gene (e.g., the regulatory region or promoter), for example, to produce a reporter construct according to the present invention. An isolated nucleic acid molecule that includes a gene is not a fragment of a chromosome that includes such gene, but rather includes the coding region and regulatory regions associated with the gene, but no additional genes naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. Preferably, an isolated nucleic acid molecule of the present invention is produced using recombinant DNA technology (e.g., polymerase chain reaction (PCR) amplification, cloning) or chemical synthesis.

The minimum size of a nucleic acid molecule or polynucleotide of the present invention is a size sufficient to encode a protein having a desired biological activity, sufficient to form a probe or oligonucleotide primer that is capable of forming a stable hybrid with the complementary sequence of a nucleic acid molecule encoding the natural protein (e.g., under moderate, high or very high stringency conditions), or to otherwise be used as a target in an assay or in any therapeutic method discussed herein. If the polynucleotide is an oligonucleotide probe or primer, the size of the polynucleotide can be dependent on nucleic acid composition and percent homology or identity between the nucleic acid molecule and a complementary sequence as well as upon hybridization conditions per se (e.g., temperature, salt concentration, and formamide concentration). The minimum size of a polynucleotide that is used as an oligonucleotide probe or primer is at least about 5 nucleotides in length, and preferably ranges from about 5 to about 50 or about 500 nucleotides or greater (1000, 2000, etc.), including any length in between, in whole number increments (i.e., 5, 6, 7, 8, 9, 10, . . . 33, 34, . . . 256, 257, . . . 500 . . . 1000 . . . ), and more preferably from about 10 to about 40 nucleotides, and most preferably from about 15 to about 40 nucleotides in length. In one aspect, the oligonucleotide primer or probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a portion of a protein-encoding sequence or a nucleic acid sequence encoding a full-length protein.

In one embodiment, the polynucleotide probes are conjugated to detectable markers. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Preferably, the polynucleotide probes are immobilized on a substrate.

In one embodiment, the polynucleotide probes are hybridizable array elements in a microarray or high density array. Nucleic acid arrays are well known in the art and are described for use in comparing expression levels of particular genes of interest, for example, in U.S. Pat. No. 6,177,248, which is incorporated herein by reference in its entirety. Nucleic acid arrays are suitable for quantifying a small variations in expression levels of a gene in the presence of a large population of heterogeneous nucleic acids. Knowing the identity of the genes set forth by the present invention, nucleic acid arrays can be fabricated either by de novo synthesis on a substrate or by spotting or transporting nucleic acid sequences onto specific locations of substrate. Nucleic acids are purified and/or isolated from biological materials, such as a bacterial plasmid containing a cloned segment of sequence of interest. It is noted that all of the genes identified by the present invention have been previously sequenced, at least in part, such that oligonucleotides suitable for the identification of such nucleic acids can be produced. The database accession number for each of the genes identified by the present inventors is provided in the tables of the invention. Suitable nucleic acids are also produced by amplification of template, such as by polymerase chain reaction or in vitro transcription.

One of skill in the art will appreciate that an enormous number of array designs are suitable for the practice of this invention. An array will typically include a number of probes that specifically hybridize to the sequences of interest. In addition, in a preferred embodiment, the array will include one or more control probes. The high-density array chip includes “test probes.” Test probes could be oligonucleotides having a minimum or maximum length as described above for other oligonucleotides. In another preferred embodiments, test probes are double or single strand DNA sequences. DNA sequences are isolated or cloned from natural sources or amplified from natural sources using natural nucleic acids as templates, or produced synthetically. These probes have sequences complementary to particular subsequences of the genes whose expression they are designed to detect. Thus, the test probes are capable of specifically hybridizing to the target nucleic acid they are to detect.

Another embodiment of the present invention relates to a plurality of antibodies, or antigen binding fragments thereof, for the detection of the expression of genes regulated in peripheral blood cells in patients with PAH. The plurality of antibodies, or antigen binding fragments thereof, consists of antibodies, or antigen binding fragments thereof, that selectively bind to proteins encoded by genes that are regulated in peripheral blood cells in patients with PAH, and that can be detected as protein products using antibodies. In addition, the plurality of antibodies, or antigen binding fragments thereof, comprises antibodies, or antigen binding fragments thereof, that selectively bind to proteins or portions thereof (peptides) encoded by any of the genes from the tables provided herein. In one aspect, the plurality of antibodies, antigen binding fragments thereof, or antigen binding peptides consists of at least two antibodies, antigen binding fragments thereof, or antigen binding peptides, each of which selectively binds to a protein encoded by a gene comprising, or expressing a transcript comprising, a nucleic acid sequence selected from the group consisting of SEQ ID NOs:1-128.

According to the present invention, a plurality of antibodies, or antigen binding fragments thereof, refers to at least 2, and more preferably at least 3, and more preferably at least 4, and more preferably at least 5, and more preferably at least 6, and more preferably at least 7, and more preferably at least 8, and more preferably at least 9, and more preferably at least 10, and so on, in increments of one, up to any suitable number of antibodies, or antigen binding fragments thereof, including antibodies representing all of the genes described herein (e.g., 128) or more, such as 500, or at least 1000 antibodies, or antigen binding fragments thereof.

According to the present invention, the phrase “selectively binds to” refers to the ability of an antibody, antigen binding fragment or binding partner (antigen binding peptide) to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

Limited digestion of an immunoglobulin with a protease may produce two fragments. An antigen binding fragment is referred to as an Fab, an Fab′, or an F(ab′)₂ fragment. A fragment lacking the ability to bind to antigen is referred to as an Fc fragment. An Fab fragment comprises one arm of an immunoglobulin molecule containing a L chain (V_{L}+C_{L} domains) paired with the V_{H} region and a portion of the C_{H} region (CH1 domain). An Fab′ fragment corresponds to an Fab fragment with part of the hinge region attached to the CH1 domain. An F(ab′)₂ fragment corresponds to two Fab′ fragments that are normally covalently linked to each other through a di-sulfide bond, typically in the hinge regions.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

Finally, any of the genes of this invention, or their RNA or protein products, can serve as targets for therapeutic strategies. For example, regulatory compounds that regulate (e.g., upregulate or downregulate) the expression and/or biological activity of a target gene or its expression product (whether the product is intracellular, membrane or secreted), can be identified and/or designed using the genes described herein. Alternatively, through the identification of particular genes that are highly regulated in patients with PAH, one can use such genes and their products to further investigate the molecular or biochemical mechanisms associated with the development and progression of PAH and then design or establish assays to identify therapeutic compounds that affect the molecular or biochemical mechanism with the goal of providing a therapeutic benefit to the patient.

For example, the present inventors have selected two genes from the genes that distinguish PAH from normal individuals, and one gene from the genes that were differentially expressed between patients with IPHA and s-PAH, for their perceived biologic interest. Adrenomedullin (represented herein by SEQ ID NO:94; increased in PAH vs. normal) is a potent pulmonary vascular vasodilator (59). Plasma levels of adrenomedullin are elevated in idiopathic and secondary forms of pulmonary hypertension and its use as a therapeutic inhalational agent in the treatment of PAH is currently under investigation (58, 60, 61). Endothelial cell growth factor-1 (represented herein by SEQ ID NO:91; increased in PAH vs. normal) is a potent angiogenic factor and is increased in expression in a number of malignancies (62, 63, 64). As the plexiform lesion of PAH is composed of an abnormal proliferating endothelial cell phenotype, an association between patients with PAH and increased expression of ECGF-1 is of significant interest. Furthermore, the differential expression of HVEM (represented herein by SEQ ID NO:102) between patients with IPAH and s-PAH (increased in s-PAH vs. IPAH) is potentially relevant in the context of the inventors' recent reports of an association between IPAH and HHV-8 infection (23).

For example, one embodiment of the present invention relates to methods for identifying compounds that regulate the expression or activity of at least one of the biomarkers described herein. Preferably, such compounds can be used to further study mechanisms associated with PAH or more preferably, serve as a therapeutic agent for use in the treatment or prevention of at least one symptom or aspect of PAH, or as a lead compound for the development of such a therapeutic agent. Once a biomarker has been identified as a target according to the present invention, an assay can be used for screening and selecting a chemical compound or a biological compound having regulatory activity as a candidate reagent or therapeutic based on the ability of the compound to regulate the expression or activity of the target biomarker. Reference herein to regulating a target, can refer to one or both of regulating transcription of a target gene and regulating the translation and/or activity of its corresponding expression product. Such a compound can be referred to herein as therapeutic compound, in one embodiment. For example, a cell line that naturally expresses the gene of interest or has been transfected with the gene (or suitable portions or derivatives thereof for assaying putative regulatory compounds) or other recombinant nucleic acid molecule encoding the protein of interest is incubated with various compounds, also referred to as candidate compounds, test compounds, or putative regulatory compounds. A regulation of the expression of the gene of interest or regulation of the activities of its encoded product (e.g., biological activity) may be used to identify a therapeutic compound. Therapeutic compounds identified in this manner can then be re-tested, if desired, in other assays to confirm their activities with regard to the target biomarker or a cellular or other activity related thereto.

In the method of the invention, the identification of compounds that increase the expression or activity of those biomarkers identified herein that are downregulated in peripheral blood cells of patients with PAH as compared to peripheral blood cells of normal controls, are predicted to be useful as therapeutic reagents or lead compounds therefore in the prevention and treatment of PAH′. Similarly, the identification of compounds that decrease the expression or activity of those biomarkers identified herein that are upregulated in peripheral blood cells of patients with PAH as compared to peripheral blood cells of normal controls, are predicted to be useful as therapeutic reagents or lead compounds therefore in the prevention and treatment of PAH.

For example one embodiment of the present invention relates to a method of using the differentially expressed genes described herein or the proteins encoded thereby (i.e., the biomarkers of the invention) as a target to identify a regulatory compound for regulation of a biological function associated with that gene or protein. Such a method can include the steps of: (a) contacting a test compound with a cell that expresses the target biomarker or a useful portion thereof (i.e., useful being any portion of a gene, transcript or protein that can be used to identify a compound as discussed herein); and (b) identifying compounds that regulate the expression or activity of the gene or protein.

In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications, activities or interactions which result in a decrease in protein expression or a decrease in the activity of the protein, can be referred to as inactivation (complete or partial), down-regulation, reduced action, or decreased action or activity of a protein. Similarly, modifications, activities or interactions which result in an increase in protein expression or an increase in the activity of the protein, can be referred to as amplification, overproduction, activation, enhancement, up-regulation or increased action of a protein. The biological activity of a protein according to the invention can be measured or evaluated using any assay for the biological activity of the protein as known in the art. Such assays can include, but are not limited to, binding assays, assays to determine internalization of the protein and/or associated proteins, enzyme assays, cell signal transduction assays (e.g., phosphorylation assays), and/or assays for determining downstream cellular events that result from activation or binding of the cell surface protein (e.g., expression of downstream genes, production of various biological mediators, etc.).

According to the present invention, a biologically active fragment or homologue of a gene, nucleic acid transcript or derivative thereof, or protein maintains the ability to be useful in a method of the present invention. Therefore, the biologically active fragment or homologue maintains the ability to be used to identify regulators (e.g., inhibitors) of the native gene or protein when, for example, the biologically active fragment or homologue is expressed by a cell or used in another assay format. Therefore, the biologically active fragment or homologue has a structure that is sufficiently similar to the structure of the native gene or protein that a regulatory compound can be identified by its ability to bind to and/or regulate the expression or activity of the fragment or homologue in a manner consistent with the regulation of the native gene or protein.

Compounds to be screened in the methods of the invention include known organic compounds such as antibodies, products of peptide libraries, and products of chemical combinatorial libraries. Compounds may also be identified using rational drug design relying on the structure of the product of a gene. Such methods are known to those of skill in the art and involve the use of three-dimensional imaging software programs. For example, various methods of drug design, useful to design or select mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety.

As used herein, a mimetic refers to any peptide or non-peptide compound that is able to mimic the biological action of a naturally occurring peptide, often because the mimetic has a basic structure that mimics the basic structure of the naturally occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art.

A mimetic can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

As used herein, the term “test compound”, “putative inhibitory compound” or “putative regulatory compound” refers to compounds having an unknown or previously unappreciated regulatory activity in a particular process. As such, the term “identify” with regard to methods to identify compounds is intended to include all compounds, the usefulness of which as a regulatory compound for the purposes of regulating the expression or activity of a target biomarker or otherwise regulating some activity that may be useful in the study or treatment of PAH is determined by a method of the present invention.

In one embodiment of the invention, regulatory compounds are identified by exposing a target gene to a test compound; measuring the expression of a target; and selecting a compound that regulates (up or down) the expression of the target. For example, the putative regulatory compound can be exposed to a cell that expresses the target gene (endogenously or recombinantly). A preferred cell to use in an assay includes a mammalian cell that either naturally expresses the target gene or has been transformed with a recombinant form of the target gene, such as a recombinant nucleic acid molecule comprising a nucleic acid sequence encoding the target protein or a useful fragment thereof. Methods to determine expression levels of a gene are well known in the art.

The conditions under which a cell, cell lysate, nucleic acid molecule or protein of the present invention is exposed to or contacted with a putative regulatory compound, such as by mixing, are any suitable culture or assay conditions. In the case of a cell-based assay, the conditions include an effective medium in which the cell can be cultured or in which the cell lysate can be evaluated in the presence and absence of a putative regulatory compound. Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art. Cells are contacted with a putative regulatory compound under conditions which take into account the number of cells per container contacted, the concentration of putative regulatory compound(s) administered to a cell, the incubation time of the putative regulatory compound with the cell, and the concentration of compound administered to a cell. Determination of effective protocols can be accomplished by those skilled in the art based on variables such as the size of the container, the volume of liquid in the container, conditions known to be suitable for the culture of the particular cell type used in the assay, and the chemical composition of the putative regulatory compound (i.e., size, charge etc.) being tested. A preferred amount of putative regulatory compound(s) can comprise between about 1 nM to about 10 mM of putative regulatory compound(s) per well of a 96-well plate.

To detect expression of a target refers to the act of actively determining whether a target is expressed or not. This can include determining whether the target expression is upregulated as compared to a control, downregulated as compared to a control, or unchanged as compared to a control. Therefore, the step of detecting expression does not require that expression of the target actually is upregulated or downregulated, but rather, can also include detecting that the expression of the target has not changed (i.e., detecting no expression of the target or no change in expression of the target). Expression of transcripts and/or proteins is measured by any of a variety of known methods in the art, and such methods have been discussed previously herein. Similarly, measurement of translation of a protein includes any suitable method for detecting and/or measuring proteins from a cell or cell extract, and such methods have been described previously herein.

Designing a compound for testing in a method of the present invention can include creating a new chemical compound or searching databases of libraries of known compounds (e.g., a compound listed in a computational screening database containing three dimensional structures of known compounds). Designing can also be performed by simulating chemical compounds having substitute moieties at certain structural features. The step of designing can include selecting a chemical compound based on a known function of the compound. A preferred step of designing comprises computational screening of one or more databases of compounds in which the three dimensional structure of the compound is known and is interacted (e.g., docked, aligned, matched, interfaced) with the three dimensional structure of a target by computer (e.g. as described by Humblet and Dunbar, Animal Reports in Medicinal Chemistry, vol. 28, pp. 275-283, 1993, M Venuti, ed., Academic Press). Methods to synthesize suitable chemical compounds are known to those of skill in the art and depend upon the structure of the chemical being synthesized. Methods to evaluate the bioactivity of the synthesized compound depend upon the bioactivity of the compound (e.g., inhibitory or stimulatory).

Candidate compounds identified or designed by the above-described methods can be synthesized using techniques known in the art, and depending on the type of compound. Synthesis techniques for the production of non-protein compounds, including organic and inorganic compounds are well known in the art. For example, for smaller peptides, chemical synthesis methods are preferred. For example, such methods include well known chemical procedures, such as solution or solid-phase peptide synthesis, or semi-synthesis in solution beginning with protein fragments coupled through conventional solution methods. Such methods are well known in the art and may be found in general texts and articles in the area such as: Merrifield, 1997, Methods Enzymol. 289:3-13; Wade et al., 1993, Australas Biotechnol. 3(6):332-336; Wong et al., 1991, Experientia 47(11-12):1123-1129; Carey et al., 1991, Ciba Found Symp. 158:187-203; Plaue et al., 1990, Biologicals 18(3):147-157; Bodanszky, 1985, Int. J. Pept. Protein Res. 25(5):449-474; or H. Dugas and C. Penney, BIOORGANIC CHEMISTRY, (1981) at pages 54-92, all of which are incorporated herein by reference in their entirety. For example, peptides may be synthesized by solid-phase methodology utilizing a commercially available peptide synthesizer and synthesis cycles supplied by the manufacturer. One skilled in the art recognizes that the solid phase synthesis could also be accomplished using the FMOC strategy and a TFA/scavenger cleavage mixture. A compound that is a protein or peptide can also be produced using recombinant DNA technology and methods standard in the art, particularly if larger quantities of a protein are desired.

In another embodiment of the invention, putative regulatory compounds are identified by exposing a target to a candidate compound; measuring the binding of the candidate compound to the target; and selecting a compound that binds to the target at a desired concentration, affinity, or avidity. In a preferred embodiment, the assay is performed under conditions conducive to promoting the interaction or binding of the compound to the target. One of skill in the art can determine such conditions based on the target and the compound being used in the assay. In one embodiment, a BIAcore machine can be used to determine the binding constant of a complex between the target protein (a protein encoded by the target gene) and a natural ligand in the presence and absence of the candidate compound. For example, the target protein or a ligand binding fragment thereof can be immobilized on a substrate. A natural or synthetic ligand is contacted with the substrate to form a complex. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Contacting a candidate compound at various concentrations with the complex and monitoring the response function (e.g., the change in the refractive index with respect to time) allows the complex dissociation constant to be determined in the presence of the test compound and indicates whether the candidate compound is either an inhibitor or an agonist of the complex. Alternatively, the candidate compound can be contacted with the immobilized target protein at the same time as the ligand to see if the candidate compound inhibits or stabilizes the binding of the ligand to the target protein.

Other suitable assays for measuring the binding of a candidate compound to a target protein or for measuring the ability of a candidate compound to affect the binding of the target protein to another protein or molecule include, but are not limited to, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry. Other assays include those that are suitable for monitoring the effects of protein binding, including, but not limited to, cell-based assays such as: cytokine secretion assays, or intracellular signal transduction assays that determine, for example, protein or lipid phosphorylation, mediator release or intracellular Ca⁺⁺ mobilization.

In yet another embodiment, putative regulatory compounds are identified by exposing a target protein of the present invention (or a cell expressing the protein naturally or recombinantly) to a candidate compound and measuring the ability of the compound to inhibit or enhance a biological activity of the protein. In one embodiment, the biological activity of a protein encoded by the target gene is measured by measuring the amount of product generated in a biochemical reaction mediated by the protein encoded by the target gene. In still another embodiment, the activity of the protein encoded by the target gene is measured by measuring the amount of substrate generated in a biochemical reaction mediated by the protein encoded by the target gene. In another embodiment, a biological activity is measured by measuring a specific event in a cell-based assay, such as release or secretion of a biological mediator or compound that is regulated by the activity of the target protein, measuring intracellular signal transduction assays that determine, for example, protein or lipid phosphorylation, mediator release or intracellular Ca⁺⁺ mobilization. Preferably, the activity of the protein is measured in the presence and absence of the candidate compound, or in the presence of another suitable control compound.

In one embodiment of the invention, when the protein encoded by a target gene is an enzyme, a therapeutic compound is identified by exposing the enzyme encoded by a target gene to a test compound; measuring the activity of the enzyme encoded by the target gene in the presence and absence of the compound; and selecting a compound that down-regulates or inhibits the activity of the enzyme encoded by the target gene. Methods to measure enzymatic activity are well known to those skilled in the art and are selected based on the identity of the enzyme being tested. For example, if the enzyme is a kinase, phosphorylation assays can be used.

Preferably, methods used to identify therapeutic compounds are customized for each target gene or product. For example, if the target product is an enzyme, then the enzyme will be expressed in cell culture and purified. The enzyme will then be screened in vitro against therapeutic compounds to look for inhibition of that enzymatic activity. If the target is a non-catalytic protein, then it will also be expressed and purified. Therapeutic compounds will then be tested for their ability to regulate, for example, the binding of a site-specific antibody or a target-specific ligand to the target product.

In a preferred embodiment, therapeutic compounds that bind to target products are identified, then those compounds can be further tested in biological assays that test for other desirable characteristics and activities, such as utility as a reagent for the study of PAH or utility as a therapeutic compound for the prevention or treatment of PAH.

If a suitable therapeutic compound is identified using the methods and genes of the present invention, a composition can be formulated. A composition, and particularly a therapeutic composition, of the present invention generally includes the therapeutic compound and a carrier, and preferably, a pharmaceutically acceptable carrier. According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. A suitable in vitro, in vivo or ex vivo site is preferably a pulmonary tissue or a cell that is associated with or travels to a pulmonary tissue. Preferred pharmaceutically acceptable carriers are capable of maintaining a compound, a protein, a peptide, nucleic acid molecule or mimetic (drug) in a form that, upon arrival of the compound, protein, peptide, nucleic acid molecule or mimetic at the target site in a culture (in the case of an in vitro or ex vivo protocol) or in patient (in vivo), the compound, protein, peptide, nucleic acid molecule or mimetic is capable of providing the desired effect at the target site.

Suitable excipients of the present invention include excipients or formularies that transport or help transport, but do not specifically target a composition to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

One type of pharmaceutically acceptable carrier includes a controlled release formulation that is capable of slowly releasing a composition of the present invention into a patient or culture. As used herein, a controlled release formulation comprises a therapeutic compound in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers include liquids that, upon administration to a patient, form a solid or a gel in situ. Preferred carriers are also biodegradable (i.e., bioerodible). When the compound is a recombinant nucleic acid molecule, suitable delivery vehicles include, but are not limited to liposomes, viral vectors or other delivery vehicles, including ribozymes. Natural lipid-containing delivery vehicles include cells and cellular membranes. Artificial lipid-containing delivery vehicles include liposomes and micelles. A delivery vehicle of the present invention can be modified to target to a particular site in a patient, thereby targeting and making use of a therapeutic compound at that site. Suitable modifications include manipulating the chemical formula of the lipid portion of the delivery vehicle and/or introducing into the vehicle a targeting agent capable of specifically targeting a delivery vehicle to a preferred site, for example, a preferred cell type. Other suitable delivery vehicles include gold particles, poly-L-lysine/DNA-molecular conjugates, and artificial chromosomes.

A compound or composition can be delivered to a cell culture or patient by any suitable method. Selection of such a method will vary with the type of compound being administered or delivered (i.e., compound, protein, peptide, nucleic acid molecule, or mimetic), the mode of delivery (i.e., in vitro, in vivo, ex vivo) and the goal to be achieved by administration/delivery of the compound or composition. According to the present invention, an effective administration protocol (i.e., administering a composition in an effective manner) comprises suitable dose parameters and modes of administration that result in delivery of a composition to a desired site (i.e., to a desired cell) and/or in the desired regulatory event.

Administration routes include in vivo, in vitro and ex vivo routes. In vivo routes include, but are not limited to, oral, nasal, intratracheal injection, inhaled, transdermal, rectal, and parenteral routes. Preferred parenteral routes can include, but are not limited to, subcutaneous, intradermal, intravenous, intramuscular and intraperitoneal routes. Intravenous, intraperitoneal, intradermal, subcutaneous and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art. Direct injection techniques are particularly useful for suppressing graft rejection by, for example, injecting the composition into the transplanted tissue, or for site-specific administration of a compound, such as at the site of a tumor. Ex vivo refers to performing part of the regulatory step outside of the patient, such as by transfecting a population of cells removed from a patient with a recombinant molecule comprising a nucleic acid sequence encoding a protein according to the present invention under conditions such that the recombinant molecule is subsequently expressed by the transfected cell, and returning the transfected cells to the patient. In vitro and ex vivo routes of administration of a composition to a culture of host cells can be accomplished by a method including, but not limited to, transfection, transformation, electroporation, microinjection, lipofection, adsorption, protoplast fusion, use of protein carrying agents, use of ion carrying agents, use of detergents for cell permeabilization, and simply mixing (e.g., combining) a compound in culture with a target cell.

In the method of the present invention, a therapeutic compound, as well as compositions comprising such compounds, can be administered to any organism, and particularly, to any member of the Vertebrate class, Mammalia, including, without limitation, primates, rodents, livestock and domestic pets. Livestock include mammals to be consumed or that produce useful products (e.g., sheep for wool production). Preferred mammals to protect include humans. Typically, it is desirable to obtain a therapeutic benefit in a patient. A therapeutic benefit is not necessarily a cure for a particular disease or condition, but rather, preferably encompasses a result which can include alleviation of the disease or condition, elimination of the disease or condition, reduction of a symptom associated with the disease or condition, prevention or alleviation of a secondary disease or condition resulting from the occurrence of a primary disease or condition, and/or prevention of the disease or condition. As used herein, the phrase “protected from a disease” refers to reducing the symptoms of the disease; reducing the occurrence of the disease, and/or reducing the severity of the disease. Protecting a patient can refer to the ability of a composition of the present invention, when administered to a patient, to prevent a disease from occurring and/or to cure or to alleviate disease symptoms, signs or causes. As such, to protect a patient from a disease includes both preventing disease occurrence (prophylactic treatment) and treating a patient that has a disease (therapeutic treatment) to reduce the symptoms of the disease. A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

The following examples are provided for the purpose of illustration and are not intended to limit the scope of the present invention.

### EXAMPLES

The following example describes the identification of the biomarkers useful in the present invention.

Subjects and Blood Collection

Blood donors were volunteers, as approved by the institutional human-subjects review board (COMIRB protocol number 00-605). All subjects gave informed consent. The microarray cohort of subjects were composed of 15 patients diagnosed with PAH (7 patients diagnosed with IPAH, 8 diagnosed with PAH related to a secondary cause, s-PAH) and 6 normal volunteers (Table 1). The prospective cohort of patients were composed of 14 patients with PAH (4 patients with IPAH and 10 patients with s-PAH) and 6 normal volunteers (Table 2). The diagnosis of IPAH was established using the algorithm developed by the IPAH NIH registry (25). Patients were excluded from study if there was evidence of other active disease process unrelated to PAH within the preceding 30 days, (i.e. systemic infection, bleeding diathesis etc.) Blood recovered from a peripheral venipuncture was collected between 1 pm and 4 pm into vacutainer tubes containing EDTA. All blood was drawn from a peripheral vein through a 21 gauge needle. All specimens were processed within 2 hours of collection. The enrolled patients had previously undergone right heart catheterization to confirm the diagnosis and severity of the pulmonary hypertension. The peripheral blood draws were performed at least 14 days after the cardiac catheterization and in the majority of cases the blood draw occurred more then 60 days after the catheterization. Echocardiography was performed within 60 days of the blood draw to confirm the persistent elevation in pulmonary arterial pressure. The blood from these patients was compared to blood from 6 normal volunteers. The patients and controls were age matched. The patients with IPAH compared to s-PAH were matched by disease severity (mean pulmonary artery pressure, cardiac output and pulmonary vascular resistance).

The results from the gene microarrays were confirmed by q-PCR retrospectively on a subset of patients from the microarray cohort (5 patients with PAH and 3 normal controls) and prospectively on second group of PAH patients and normal controls (14 patients with PAH and 6 normal controls) (Table 2).

Isolation of PBMCs:

Four ml of peripheral blood were collected in tubes containing EDTA. The blood was diluted in three volumes of PBS+2 mM EDTA+0.5% BSA. The mononuclear cell layer, which includes monocytes/macrophages, B and T lymphocytes and Natural Killer (NK) cells, was isolated via density gradient centrifugation (Hisotpaque 1077, 1200 rpm for 30 min). The purity of the mononuclear cell layer was assessed by Coulter counter on a representative population of the patients (8 patients with PAH and 6 normal controls), and determined to comprise greater then 90% mononuclear cells.

Microarray Data Generation:

Sample preparation, RNA isolation, and high-density oligonucleotide array hybridization and scanning were performed as described previously (38). Fluorescence intensities were quantified using the Affymetrix Microarray Suite 5.0 statistical algorithm with default parameters for the array type utilized in this study (Affymetrix HuFL). Tabular gene expression data is published online in Gene Expression Omnibus, submission GSE703.

Quantitative RT PCR:

RNA was extracted from PBMCs using the RNAeasy kit (Quiagen, CA). Primers and probes were obtained from Applied Biosystems Assays on Demand (Foster City, Calif.). All reactions were performed on a Gene Amp 5700 sequence detector, (Applied Biosystems, Foster City, Calif.) using the conditions recommended by the manufacturer. Standard curves were created using cloned PCR products in concentrations ranging from 0.1 ng to 0.0001 ng/μl. The inventors confirmed the absence of nonspecific amplification by examining PCR products by agarose gel electrophoresis. Standard and experimental samples were run in triplicate and the results averaged. All results were standardized to expression of β-actin. Genes assayed by q-PCR included endothelial cell growth factor-1 (ECGF-1), adrenomedullin (ADM) and tumor necrosis factor receptor super family 14 (TNFRSF14; also referred to as Herpesvirus entry mediator (HVEM)). The primers and probes for q-PCR were purchased from Assay on Demand (Applied Biosystems, Foster City, Calif.).

Data Analysis:

Patient Demographics:

Statistical analysis of demographic data was performed with the Prism version 3.0 for windows (GraphPad Software, SD, CA). One way ANOVA was used to compare patient ages. Unpaired t-tests were used to compare mean PA pressures, cardiac output and PVR between patients with PAH. Unpaired t-tests with a Mann-Whitney correction were used to analyze q-PCR results.

Microarray Analysis:

The array data were analyzed with BRB ArrayTools v3.0.2E developed by Dr. Richard Simon and Amy Tan. The initial dataset consisted of 6086 gene measurements for each of the 21 samples. Genes whose expression was not reliably detected (i.e. had an “absolute call” of “present” or “marginal”, details in the Affymetrix Signal Algorithm Description Document white paper) in at least 11 of the 21 samples were excluded, resulting in the inclusion of 2906 gene expression measurements in 21 samples. These measurements were mean-centered and analyzed using the clustering, class comparison and class prediction functions of BRB ArrayTools. The Gene Ontology™ (GO) analysis was conducted using GenMAPP and MAPPFinder (Doniger et al., 2003. MAPPFinder: using Gene Ontology and GenMAPP to create a global gene-expression profile from microarray data. Genome Biol. 4:R7). The z-score assigned to each category by MAPPFinder reflects the degree to which the differential expression of genes in that category was greater than that expected by chance. A high, positive z-score indicates that a large number of genes in that category are differentially expressed between the compared conditions.

Results:

Patient Characteristics:

Table 1 displays the characteristics of the patients who's PBMCs underwent microarray analysis. There was not a statistically significant difference between patients with PAH and normal individuals by age (50.2+/−3.5 years, n=15 vs. 39.0+/−1.9 years, n=6 mean+/−SEM, p≧0.05). The patients with IPAH and s-PAH were not significantly different in terms of mean PA pressure (60.3+/−6.0 mmHg, n=7 vs. 49.8+/−1.7 mmHg, n=8, mean+/−SEM, p≧0.09), cardiac output (3.77+/−0.78 L/min, n=7 vs. 4.46+/−0.5 L/min, n=7 mean+/−SEM, p≧0.4) or PVR (18.2+/−4.0 wood units, n=5 vs. 10.1+/−2.5 wood units, n=5, mean+/−SEM, p≧0.13).

TABLE 1

| | | | | PA mmHg | | PVR | | |
|---|---|---|---|---|---|---|---|---|
| Patient | | Age | | (sys/dias, | CO | (wood | | |
| Code | Diagnosis | years | Sex | mean) | L/min | units) | Vasoresponsive | Rx |
| 01 PAH | PPH | 56 | F | 78/33 49 | 2.6 | 14.0 | No | PGI2 |
| 02 PAH | PPH | 51 | F | 58/28 40 | 1.8 | 21.7 | No | PGI2 |
| 03 PAH | PPH | 43 | F | 84/34 54 | 6.2 | 16.3 | No | PGI2 |
| 04 PAH | PPH | 50 | F | 82/41 56 | 2.6 | * | No | PGI2 |
| 05 PAH | PPH | 45 | F | 90/48 64 | 3.5 | * | No | PGI2 |
| 06 PAH | PPH | 27 | M | 123/64 89 | 2.5 | 32.4 | No | PGI2 |
| 07 PAH | PPH | 27 | M | 84/50 70 | 7.2 | 8 | No | PGI2 |
| 08 PAH | Portal Htn. | 44 | F | 75/33 50 | 4.5 | 7.4 | Yes | Ca++ |
| 09 PAH | CREST | 69 | F | 70/30 45 | 4.8 | * | Yes | Ca++ |
| 10 PAH | ILD | 67 | M | 64/31 44 | 2 | 20 | No | PGI2 |
| 11 PAH | PE | 50 | M | 90/40 58 | 6.2 | 9.3 | Yes | Ca++ |
| 12 PAH | CREST | 42 | F | 80/41 54 | 4 | * | No | PGI2 |
| 13 PAH | CREST | 50 | F | 76/36 47 | 4.2 | 9.8 | No | Bosentan |
| 14 PAH | Phen/fen | 65 | F | 73/33 47 | 5.5 | 5.7 | Yes | Ca++ |
| 15 PAH | Phen/fen | 67 | F | 90/35 53 | * | * | No | PGI2 |
| 01 Nor | Normal | 43 | F | * | * | * | * | * |
| 02 Nor | Normal | 43 | F | * | * | * | * | * |
| 03 Nor | Normal | 44 | M | * | * | * | * | * |
| 04 Nor | Normal | 35 | M | * | * | * | * | * |
| 05 Nor | Normal | 35 | M | * | * | * | * | * |
| 06 Nor | Normal | 34 | M | * | * | * | * | * |

(* = no data available,
CREST = calcinosis,
Raynaud's phenomenon, esophageal dismotility, sclerodactyly, telengectasias,
ILD = intersitial lung disease,
PE = chronic pulmonary thromboemboloic disease,
Phen/fen = exposure to the anorexigens phenteramine/fenfluramine,
PGI2 = treatment with intravenous epoprostenol,
Rx = therapy received at the time of blood draw,
Ca++ = treatment with calcium channel blockers,
vasoresponsive indicates >20% decrease in mean PA pressure and >25% decrease in PVR after an acute vasodilator trial)

Table 2 displays the characteristics of patients who underwent quantitative PCR for prospective confirmation of the microarray data. The patients with PAH and the normal volunteers were matched by age (53.6+/−3.6 years, n=14 vs. 47.0+/−5.3 years, n=6, mean+/−SEM, p≧0.3). The patients with IPAH and s-PAH were not significantly different in terms of mean PA pressure (53.0+/−7.3 mmHg, n=4, vs. 60.3+/−6.1 mmHg, n=8, mean+/−SEM, p≧0.4), cardiac output (3.5+/−0.41 L/min, n=4 vs. 2.7+/−0.54 L/min, n=8, mean+/−SEM, p≧0.3) or PVR (10.5+/−3.6, n=4 vs. 15.0+/−1.7, n=8, mean+/−SEM, p≧0.23).

TABLE 2

| | | | | | PVR | | |
|---|---|---|---|---|---|---|---|
| | Age | | PA mmHg | CO | Wood | | |
| Diagnosis | years | Sex | (sys/dias/mean) | L/min | Units | vasoresponsive | Rx |
| PPH^{01PAH} | 56 | F | 78/33 49 | 2.6 | 14 | No | PGI2 |
| PPH^{02PAH} | 51 | F | 58/28 40 | 1.8 | 21.7 | No | PGI2 |
| PPH^{05PAH} | 45 | F | 120/60 82 | 3.4 | * | No | PGI2 |
| PPH^{06PAH} | 27 | M | 123/64 89 | 2.5 | 32.4 | No | PGI2 |
| PPH | 41 | F | 95/49 65 | 2.7 | 17.1 | No | PGI2 |
| PPH | 47 | F | 63/28 47 | 4.6 | 2.7 | No | PGI2 |
| FPPH | 30 | F | 91/47 65 | 3 | 16.3 | No | None |
| PPH | 44 | F | 47/25 35 | 3.6 | 5.8 | No | PGI2 |
| CREST | 54 | F | 70/26 44 | 4.1 | 16 | No | PGI2 |
| CREST | 50 | F | 67/32 45 | 1.4 | 13.5 | No | PGI2 |
| CREST | 66 | M | 86/39 55 | 1.7 | 11.7 | No | PGI2 |
| CREST | 53 | F | 117/52 78 | 2.7 | * | Yes | Ca++ |
| CREST | 66 | M | 69/35 49 | 2.9 | 13.3 | Yes | Ca++ |
| CREST | 75 | F | 144/58 92 | 2.2 | 20 | No | PGI2 |
| ILD^{10PAH} | 67 | M | 64/31 44 | 2 | 20 | No | PGI2 |
| PE^{11PAH} | 50 | M | 90/40 58 | 6.2 | 9.3 | Yes | Ca++ |
| PE | 56 | M | 99/53 68 | 1.2 | 21.8 | No | Coumadin |
| Portal Htn | 52 | M | 90 systolic | * | * | * | None |
| COPD | 77 | M | 70 systolic | * | * | * | O2 |
| Phen/fen^{15PAH} | 67 | F | 90/35 53 | * | * | No | PGI2 |
| HIV-1 | 40 | F | 81/35 52 | 5.7 | 8.9 | No | None |
| Normal | 64 | F | * | * | * | * | * |
| Normal | 32 | F | * | * | * | * | * |
| Normal | 48 | F | * | * | * | * | * |
| Normal | 35 | M | * | * | * | * | * |
| Normal | 43 | F | * | * | * | * | * |
| Normal^{06Nor} | 34 | M | * | * | * | * | * |
| Normal | 60 | M | * | * | * | * | * |

* = no data available,
CREST = calcinosis, Raynaud's phenomenon, esophageal dismotility, sclerodactyly, telengectasias,
ILD = intersitial lung disease,
PE = chronic pulmonary thromboemboloic disease,
HIV = infection with HIV-1,
COPD = chronic obstructive pulmonary disease,
PGI2 = treatment with intravenous epoprostenol,
Rx = therapy received at the time of blood draw,
Ca++ = treatment with calcium channel blockers,
02 = treatment with chronic oxygen therapy.

Microarray Analysis

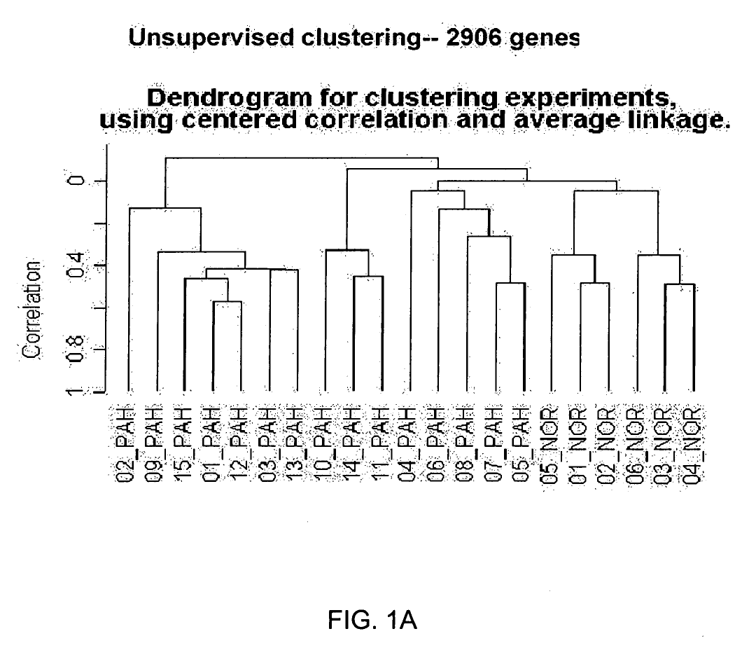

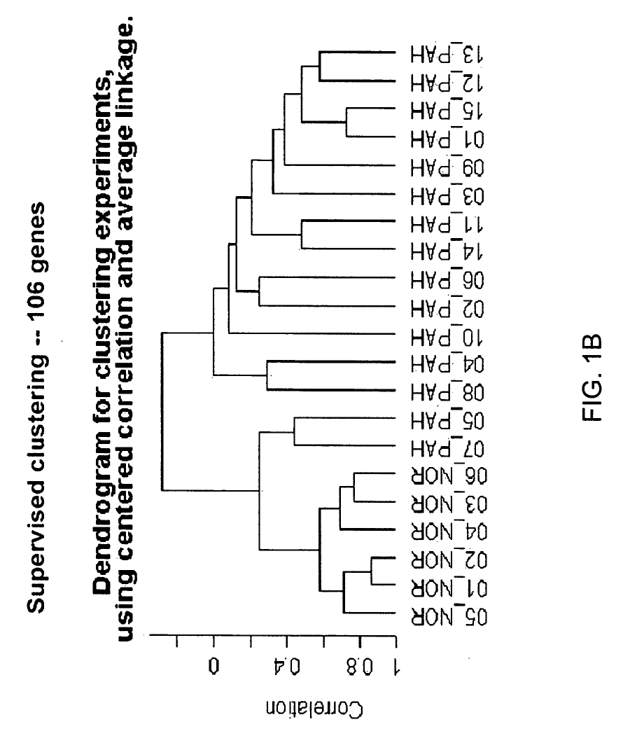

Microarray data were examined first in an unsupervised mode, utilizing expression values for all 2906 probe sets present in the majority of samples. Clustering of these data is shown in FIG. 1A, which shows that the non-PAH samples are more closely related to one another than to the PAH samples. The robustness of this grouping is high (0.931) when perturbed data is re-clustered. A 106 gene expression signature (101 of these genes are represented in Table 3, accounting for the removal of five genes with poorly annotated probe sets) which supported this segregation of normal and PAH samples was generated using a two sample t test and a conservative p-value cutoff (0.001) for expression differences supporting the class assignment. Permutation testing of the resulting 106 gene signature suggests that this list contains fewer than 2 false discoveries. Supervised clustering of the samples using this signature results in a more robust (0.971) grouping of the normal volunteers (FIG. 1B). Most (96 of 106) of the gene expression values in this signature have a higher mean value in the PAH samples relative to the non-PAH samples (data not shown).

The utility of PBMC gene expression data for sample discrimination was evaluated in two separate prediction protocols. In the first protocol, a gene expression profile was developed from the first 14 patient and volunteer samples evaluated. This profile was used to predict the class membership of 7 subsequent samples (05 Nor, 06 Nor, 04 PAH, 06 PAH, 07 PAH, 08 PAH, 13 PAH). In each case the correct prediction was made with all of the available prediction algorithms (data not shown). Due to the limited size of the patient group a second, “leave-one-out” protocol was employed for prediction accuracy cross-validation; this has the advantage of using the data more efficiently. In the leave-one-out cross-validation, each patient or volunteer sample was excluded from the data set sequentially, and the remaining 20 samples were used to build a gene expression profile discriminating between the two classes, and the resulting profile was used to predict the class of the left-out specimen. In each of the 21 iterations an independent expression profile was computed, and the prediction of the left-out specimen was completed with 95% or 100% accuracy using the available algorithms (see Table 7). The p-values for each of the predictors are estimated to be ≦0.002 based on 2000 random permutations.

Table 4 shows a partial list of genes with significant differences in the expression between patients diagnosed with PAH and normal controls. These are genes are deemed by the present inventors to be of high potential biologic interest.

In addition to the comparisons of normal and PAH, the inventors conducted a comparison of PBMC gene expression within the PAH patient population of 7 IPAH and 8 s-PAH samples. Comparison of these two groups with the class comparison protocol did not reveal a statistically significant pattern of gene expression discriminating between these two groups. However, some individual genes did attain a nominal significance in this class comparison: 28 genes are significant at the α=0.01 level, 178 are significant at the a=0.05 level (data not shown). However, both sets of nominally significant genes failed to sustain significance upon permutation testing, resulting in an estimated 20% probability that either of these sets would have little predictive value in a larger cohort.

q-PCR Results:

PAH vs. Normal

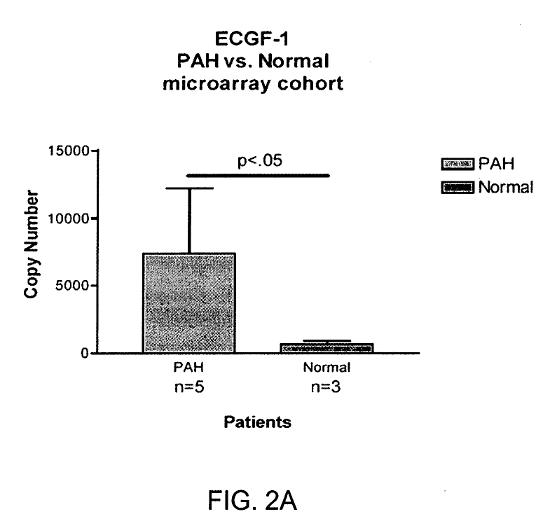

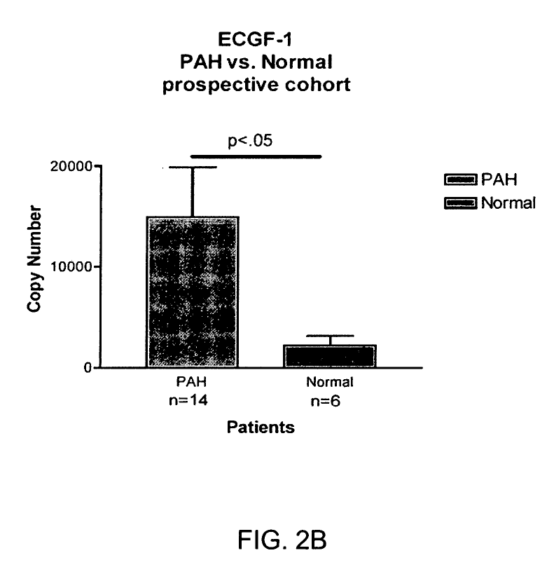

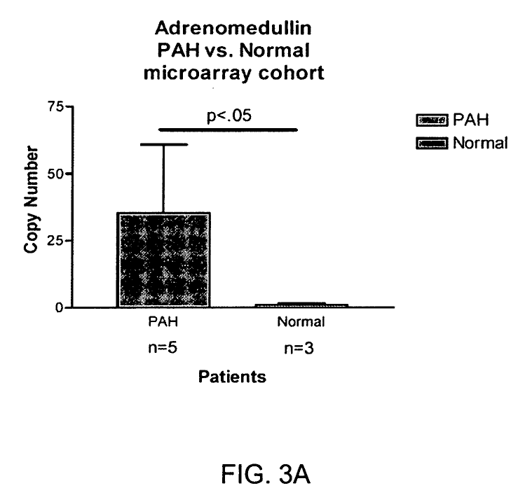

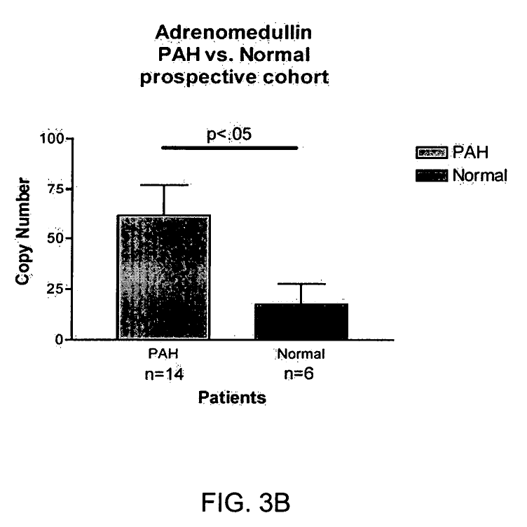

Two genes identified through microarray analysis to distinguish patients with PAH vs. normal individuals were selected for further investigation using q-PCR. The genes were selected both for their ability to discriminate between groups by microarray and their perceived biologic interest. These genes were endothelial cell growth factor 1 (ECGF-1) (p=0.0008) and adrenomedullin (ADM) (p=0.0008). Quantitative PCR was performed on the PBMC samples of a subset of the patients who had undergone microarray analysis (patients PAH 1, PAH 2, PAH 5, PAH 6, PAH 10, PAH 11, PAH 15, Normal 2, Normal 3, Normal 6). As predicted by the microarray data there was a significant difference in the expression of these genes between patients with PAH compared to normal controls (ECGF-1, 14930+/−4912 n=5 vs. 2175+/−963, n=3, mean+/−SEM, p≦0.04) (ADM, 35.4+/−25 n=5 vs. 1.1+/−0.5 n=3, mean+/−SEM, p≦0.04) (FIGS. 2A and 3A). Quantitative PCR for these 2 genes was then performed on a second prospective cohort of normal individuals and patients with PAH. Again, a significant difference of expression of ECGF-1 and ADM was detected in PAH vs. normal controls in the direction predicted by the microarray (ECGF-1, 14930+/−4912 n=14 vs. 2175+/−963 n=6, mean+/−SEM, p≦0.05) (ADM, 61.5+/−15.8 n=14 vs. 17.5+/−10.4 n=6 mean+/−SEM, p≦0.03) (FIGS. 2B and 3B).

IPAH Versus s-PAH

Re-analysis of the microarray data using a supervised class comparison algorithm identified a list of 28 genes (Table 5) which were differentially expressed in patients with IPAH vs. s-PAH. One of these genes, tumor necrosis factor receptor superfamily, member 14 (TNRSF14), also called herpesvirus entry mediator (HVEM) (p=0.007) was selected for analysis on our prospective cohort of patients by q-PCR.

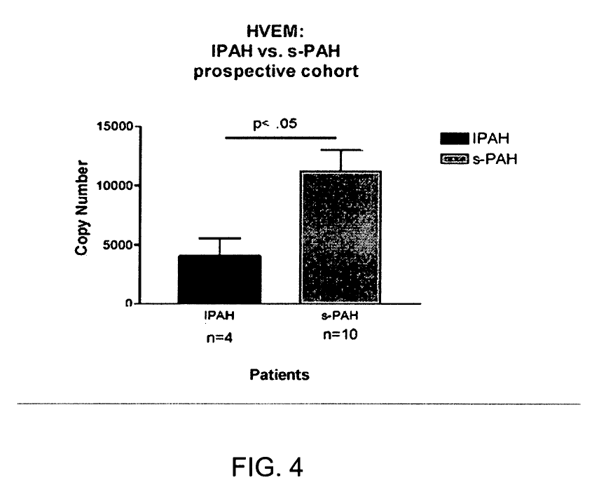

Quantitative PCR confirmed a significant difference in the expression of HVEM in the PBMCs of patients with IPAH compared to patients diagnosed with s-PAH (5157+/−1248 n=9 vs. 10410+/−1412 n=7, mean+/−SEM, p≦0.05, expressed as copy number) using this method (FIG. 4).

Gene Ontology Analysis:

In order to discover classes of genes which were involved in PAH, GenMAPP and MAPPfinder were used, a less stringent statistical criteria for the identification of groups of genes was employed than had been used for the identification of individual genes. This software calculates a standardized difference score (z-score) for each gene category, comparing the number of observed changes in a category to the number expected in that category by chance. Table 6 lists discriminating genes by their described Gene Ontology (GO) category. To be included in this list the genes had to have a z score >3.0 and a minimum of 6 genes had to be differentially expressed in the involved GO category. A significant number of genes in the GO categories of inflammatory response, stress response, cytochrome c oxidase, lysosome and intracellular signaling cascade were identified as being differentially expressed.

TABLE 3

| | | | | Fold | | | |
|---|---|---|---|---|---|---|---|
| | | Mean | Mean | Change | | | |
| | Parametric | Intensity | Intensity | PAH/ | Gene | Sequence | |
| Probe set ID | p-value | Normal | PAH | Normal | Symbol | Identifier | Gene Name |
| X57346_at | 1.50E−06 | 2004.88 | 4033.92 | 0.497 | YWHAB | SEQ ID NO: 1 | tyrosine 3-monooxygenase/tryptophan 5- |
| | | | | | | | monooxygenase activation protein, beta |
| | | | | | | | polypeptide |
| M97936_at | 1.50E−06 | 392.72 | 1397.49 | 0.281 | STAT1 | SEQ ID NO: 2 | signal transducer and activator of transcription 1, |
| | | | | | | | 91 kDa |
| U09587_at | 2.30E−06 | 482.51 | 882.16 | 0.547 | GARS | SEQ ID NO: 3 | glycyl-tRNA synthetase |
| U44772_at | 2.70E−06 | 1201.31 | 2274.17 | 0.528 | PPT1 | SEQ ID NO: 4 | palmitoyl-protein thioesterase 1 (ceroid- |
| | | | | | | | lipofuscinosis, neuronal 1, infantile) |
| D31797_at | 2.80E−06 | 207.81 | 126.83 | 1.639 | CD40LG | SEQ ID NO: 5 | |
| U51478_at | 2.90E−06 | 1709.98 | 3277.46 | 0.522 | ATP1B3 | SEQ ID NO: 6 | ATPase, Na+/K+ transporting, beta 3 polypeptide |
| X60003_s_at | 5.70E−06 | 542.44 | 304.53 | 1.781 | CREB1 | SEQ ID NO: 7 | cAMP responsive element binding protein 1 |
| M63904_at | 7.50E−06 | 370.40 | 666.75 | 0.556 | GNA15 | SEQ ID NO: 8 | guanine nucleotide binding protein (G protein), |
| | | | | | | | alpha 15 (Gq class) |
| U67156_at | 1.49E−05 | 80.91 | 175.28 | 0.462 | MAP3K5 | SEQ ID NO: 9 | mitogen-activated protein kinase kinase kinase 5 |
| U77643_at | 1.82E−05 | 833.40 | 1921.39 | 0.434 | SECTM1 | SEQ ID NO: 10 | secreted and transmembrane 1 |
| L34587_at | 2.05E−05 | 906.16 | 1342.20 | 0.675 | TCEB1 | SEQ ID NO: 11 | transcription elongation factor B (SIII), |
| | | | | | | | polypeptide 1 (15 kDa, elongin C) |
| L23116_at | 2.83E−05 | 213.62 | 368.27 | 0.58 | GALC | SEQ ID NO: 12 | galactosylceramidase (Krabbe disease) |
| D13146_cds1_at | 3.01E−05 | 356.33 | 510.44 | 0.698 | CNP | SEQ ID NO: 13 | |
| U90313_at | 3.07E−05 | 975.51 | 1598.29 | 0.61 | GSTO1 | SEQ ID NO: 14 | glutathione-S-transferase like; glutathione |
| | | | | | | | transferase omega |
| U94586_at | 3.20E−05 | 1151.93 | 1637.03 | 0.704 | NDUFA4 | SEQ ID NO: 15 | NADH dehydrogenase (ubiquinone) 1 alpha |
| | | | | | | | subcomplex, 4, 9 kDa |
| X61587_at | 3.24E−05 | 1911.61 | 3295.48 | 0.58 | RHOG | SEQ ID NO: 16 | ras homolog gene family, member G (rho G) |
| M63835_at | 3.59E−05 | 211.06 | 819.16 | 0.258 | FCGR1A | SEQ ID NO: 17 | |
| J04173_at | 3.67E−05 | 2595.02 | 4108.09 | 0.632 | PGAM1 | SEQ ID NO: 18 | phosphoglycerate mutase 1 (brain) |
| D30755_at | 3.94E−05 | 1259.51 | 2407.52 | 0.523 | TNIP1 | SEQ ID NO: 19 | Nef-associated factor 1 |
| D31765_at | 4.16E−05 | 198.86 | 134.69 | 1.476 | POP1 | SEQ ID NO: 20 | processing of precursors 1 |
| U70451_at | 5.57E−05 | 1011.95 | 1608.31 | 0.629 | MYD88 | SEQ ID NO: 21 | myeloid differentiation primary response gene |
| | | | | | | | (88) |
| X56681_s_at | 5.61E−05 | 3429.69 | 5514.79 | 0.622 | JUND | SEQ ID NO: 22 | jun D proto-oncogene |
| J02783_at | 0.0000577 | 796.77 | 1114.37 | 0.715 | P4HB | SEQ ID NO: 23 | procollagen-proline, 2-oxoglutarate 4- |
| | | | | | | | dioxygenase (proline 4-hydroxylase), beta |
| | | | | | | | polypeptide (protein disulfide isomerase; thyroid |
| | | | | | | | hormone binding protein p55) |
| U50523_at | 6.03E−05 | 3323.21 | 5100.76 | 0.652 | ARPC2 | SEQ ID NO: 24 | actin related protein ⅔ complex, subunit 2, |
| | | | | | | | 34 kDa |
| X62320_at | 6.46E−05 | 2600.74 | 4874.93 | 0.533 | GRN | SEQ ID NO: 25 | granulin |
| Y00636_at | 7.69E−05 | 249.95 | 480.30 | 0.52 | CD58 | SEQ ID NO: 26 | CD58 antigen, (lymphocyte function-associated |
| | | | | | | | antigen 3) |
| U34877_at | 8.22E−05 | 530.47 | 1051.31 | 0.505 | BLVRA | SEQ ID NO: 27 | biliverdin reductase A |
| X59834_at | 8.61E−05 | 616.74 | 1326.00 | 0.465 | GLUL | SEQ ID NO: 28 | glutamate-ammonia ligase (glutamine synthase) |
| L76191_at | 9.01E−05 | 776.27 | 1190.19 | 0.652 | IRAK1 | SEQ ID NO: 29 | interleukin-1 receptor-associated kinase 1 |
| M92843_s_at | 9.62E−05 | 2451.83 | 5578.04 | 0.44 | ZFP36 | SEQ ID NO: 30 | zinc finger protein 36, C3H type, homolog |
| | | | | | | | (mouse) |
| U97105_at | 9.92E−05 | 564.02 | 1083.85 | 0.52 | DPYSL2 | SEQ ID NO: 31 | dihydropyrimidinase-like 2 |
| M57710_at | 0.0001028 | 2181.64 | 4470.46 | 0.488 | LGALS3 | SEQ ID NO: 32 | lectin, galactoside-binding, soluble, 3 (galectin 3) |
| U84720_at | 0.0001136 | 389.04 | 565.86 | 0.688 | RAE1 | SEQ ID NO: 33 | RAE1 RNA export 1 homolog (S. pombe) |
| M81695_s_at | 0.0001157 | 460.97 | 760.10 | 0.606 | ITGAX | SEQ ID NO: 34 | integrin, alpha X (antigen CD11C (p150), alpha |
| | | | | | | | polypeptide) |
| M24902_at | 0.000116 | 54.38 | 107.69 | 0.505 | ACPP | SEQ ID NO: 35 | acid phosphatase, prostate |
| U02570_at | 0.000134 | 1156.98 | 1751.87 | 0.66 | ARHGAP1 | SEQ ID NO: 36 | Rho GTPase activating protein 1 |
| U41387_at | 0.0001433 | 149.48 | 379.49 | 0.394 | DDX21 | SEQ ID NO: 37 | DEAD/H (Asp-Glu-Ala-Asp/His) box polypeptide |
| | | | | | | | 21 |
| U49188_at | 0.0001506 | 617.87 | 313.91 | 1.968 | TDE1 | SEQ ID NO: 38 | tumor differentially expressed 1 |
| U46751_at | 0.000151 | 3591.41 | 5325.22 | 0.674 | SQSTM1 | SEQ ID NO: 39 | sequestosome 1 |
| U73514_at | 0.0001573 | 694.02 | 1095.57 | 0.633 | HADH2 | SEQ ID NO: 40 | hydroxyacyl-Coenzyme A dehydrogenase, type II |
| U59302_at | 0.000166 | 314.51 | 849.49 | 0.37 | NCOA1 | SEQ ID NO: 41 | nuclear receptor coactivator 1 |
| L40586_at | 0.0001763 | 64.57 | 107.29 | 0.602 | IDS | SEQ ID NO: 42 | iduronate 2-sulfatase (Hunter syndrome) |
| X79882_at | 0.0001782 | 610.47 | 1229.05 | 0.497 | MVP | SEQ ID NO: 43 | major vault protein |
| U02680_at | 0.000191 | 59.82 | 121.81 | 0.491 | PTK9 | SEQ ID NO: 44 | protein tyrosine kinase 9 |
| Z50022_at | 0.000197 | 591.75 | 1110.99 | 0.533 | PTTG1IP | SEQ ID NO: 45 | pituitary tumor-transforming 1 interacting protein |
| J04182_at | 0.0002068 | 1533.14 | 2508.17 | 0.611 | LAMP1 | SEQ ID NO: 46 | lysosomal-associated membrane protein 1 |
| M94345_at | 0.0002172 | 1176.11 | 2199.39 | 0.535 | CAPG | SEQ ID NO: 47 | capping protein (actin filament), gelsolin-like |
| D10522_at | 0.0002299 | 729.08 | 1763.68 | 0.413 | MARCKS | SEQ ID NO: 48 | myristoylated alanine-rich protein kinase C |
| | | | | | | | substrate |
| M15395_at | 0.0002301 | 696.99 | 1102.31 | 0.632 | ITGB2 | SEQ ID NO: 49 | integrin, beta 2 (antigen CD18 (p95), lymphocyte |
| | | | | | | | function-associated antigen 1; macrophage |
| | | | | | | | antigen 1 (mac-1) beta subunit) |
| M32315_at | 0.0002332 | 2005.24 | 3508.12 | 0.572 | TNFRSF1B | SEQ ID NO: 50 | tumor necrosis factor receptor superfamily, |
| | | | | | | | member 1B |
| M29696_at | 0.0002634 | 4404.62 | 817.08 | 5.391 | IL7R | SEQ ID NO: 51 | interleukin 7 receptor |
| M86934_at | 0.0002744 | 146.15 | 297.04 | 0.492 | HDHD1A | SEQ ID NO: 52 | DNA segment, numerous copies, expressed |
| | | | | | | | probes (GS1 gene) |
| U17886_at | 0.0002983 | 491.14 | 748.66 | 0.656 | SDHB | SEQ ID NO: 53 | |
| U32519_at | 0.0003058 | 351.42 | 232.48 | 1.512 | G3BP | SEQ ID NO: 54 | Ras-GTPase-activating protein SH3-domain- |
| | | | | | | | binding protein |
| U31383_at | 0.0003302 | 480.00 | 1203.27 | 0.399 | GNG10 | SEQ ID NO: 55 | guanine nucleotide binding protein 10 |
| U30825_at | 0.0003338 | 859.07 | 1333.96 | 0.644 | SFRS9 | SEQ ID NO: 56 | splicing factor, arginine/serine-rich 9 |
| S83364_at | 0.000338 | 205.23 | 403.31 | 0.509 | C20orf24 | SEQ ID NO: 57 | |
| D87989_at | 0.0003534 | 552.59 | 728.76 | 0.758 | SLC35B1 | SEQ ID NO: 58 | UDP-galactose transporter related |
| X62654_ma1_at | 0.0003543 | 820.46 | 1633.97 | 0.502 | CD63 | SEQ ID NO: 59 | |
| M62831_at | 0.0003566 | 2232.08 | 4321.70 | 0.516 | IER2 | SEQ ID NO: 60 | immediate early protein |
| U70063_at | 0.00036 | 236.08 | 375.77 | 0.628 | ASAH1 | SEQ ID NO: 61 | N-acylsphingosine amidohydrolase (acid |
| | | | | | | | ceramidase) 1 |
| U03100_at | 0.0003639 | 163.00 | 258.66 | 0.63 | CTNNA1 | SEQ ID NO: 62 | catenin (cadherin-associated protein), alpha 1 |
| | | | | | | | (102 kDa |
| Z48950_at | 0.0003749 | 4803.06 | 7020.52 | 0.684 | H3F3B | SEQ ID NO: 63 | |
| L76200_at | 0.0004017 | 1665.80 | 2848.69 | 0.585 | GUK1 | SEQ ID NO: 64 | guanylate kinase 1 |
| U09578_at | 0.0004132 | 527.83 | 1062.66 | 0.497 | MAPKAPK3 | SEQ ID NO: 65 | mitogen-activated protein kinase-activated protein |
| | | | | | | | kinase 3 |
| U80040_at | 0.0004375 | 734.63 | 1113.76 | 0.66 | ACO2 | SEQ ID NO: 66 | aconitase 2, mitochondrial |
| L04270_at | 0.000449 | 271.44 | 644.84 | 0.421 | LTBR | SEQ ID NO: 67 | lymphotoxin beta receptor (TNFR superfamily, |
| | | | | | | | member 3) |
| U49869_ma1_at | 0.0004622 | 10134.38 | 15899.42 | 0.637 | UBB | SEQ ID NO: 68 | |
| J05272_at | 0.0004904 | 982.89 | 1439.32 | 0.683 | IMPDH1 | SEQ ID NO: 69 | IMP (inosine monophosphate) dehydrogenase 1 |
| U00115_at | 0.000497 | 269.86 | 633.17 | 0.426 | BCL6 | SEQ ID NO: 70 | B-cell CLL/lymphoma 6 (zinc finger protein 51) |
| M97935_s_at | 0.0004975 | 514.28 | 928.12 | 0.554 | STAT1 | SEQ ID NO: 71 | signal transducer and activator of transcription 1, |
| | | | | | | | 91 kDa |
| U45285_at | 0.000515 | 899.52 | 1721.63 | 0.522 | TCIRG1 | SEQ ID NO: 72 | T-cell, immune regulator 1, ATPase, H+ |
| | | | | | | | transporting, lysosomal V0 protein a isoform 3 |
| M13690_s_at | 0.0005443 | 84.92 | 221.81 | 0.383 | SERPING1 | SEQ ID NO: 73 | serine (or cysteine) proteinase inhibitor, clade G |
| | | | | | | | (C1 inhibitor), member 1, (angioedema, |
| | | | | | | | hereditary) |
| D87116_at | 0.0005483 | 642.52 | 1225.41 | 0.524 | MAP2K3 | SEQ ID NO: 74 | mitogen-activated protein kinase kinase 3 |
| L13943_at | 0.0005739 | 68.47 | 128.75 | 0.532 | GK | SEQ ID NO: 75 | glycerol kinase |
| U50733_at | 0.0005915 | 659.83 | 936.58 | 0.705 | DCTN2 | SEQ ID NO: 76 | dynactin 2 (p50) |
| U41766_s_at | 0.0006224 | 37.43 | 66.03 | 0.567 | ADAM9 | SEQ ID NO: 77 | a disintegrin and metalloproteinase domain 9 |
| | | | | | | | (meltrin gamma) |
| Z35491_at | 0.0006229 | 238.34 | 446.31 | 0.534 | BAG1 | SEQ ID NO: 78 | BCL2-associated athanogene |
| D16469_at | 0.0006248 | 971.82 | 1418.73 | 0.685 | ATP6AP1 | SEQ ID NO: 79 | ATPase, H+ transporting, lysosomal interacting |
| | | | | | | | protein 1 |
| U67932_s_at | 0.0006512 | 196.27 | 117.02 | 1.677 | PDE7A | SEQ ID NO: 80 | phosphodiesterase 7A |
| U01147_at | 0.0006595 | 325.96 | 485.57 | 0.671 | ABR | SEQ ID NO: 81 | active BCR-related gene |
| U29680_at | 0.0006818 | 438.24 | 1309.96 | 0.335 | BCL2A1 | SEQ ID NO: 82 | BCL2-related protein A1 |
| M31932_at | 0.000688 | 277.04 | 496.37 | 0.558 | FCGR2A | SEQ ID NO: 83 | Fc fragment of IgG, low affinity Ila, receptor for |
| | | | | | | | (CD32) |
| U80184_ma1_at | 0.0006966 | 750.36 | 1159.34 | 0.647 | FLII | SEQ ID NO: 84 | Homo sapiens FLII gene |
| X77584_at | 0.0007029 | 807.98 | 1292.03 | 0.625 | TXN | SEQ ID NO: 85 | thioredoxin |
| X60036_at | 0.0007185 | 2879.03 | 3778.17 | 0.762 | SLC25A3 | SEQ ID NO: 86 | solute carrier family 25 (mitochondrial carrier; |
| | | | | | | | phosphate carrier), member 3 |
| U03057_at | 0.0007466 | 236.72 | 415.76 | 0.569 | FSCN1 | SEQ ID NO: 87 | singed-like (fascin homolog, sea urchin) |
| | | | | | | | (Drosophila) |
| U51336_at | 0.000751 | 1060.10 | 1353.87 | 0.783 | ITPK1 | SEQ ID NO: 88 | inositol 1,3,4-triphosphate 5/6 kinase |
| U00921_at | 0.0007747 | 739.53 | 1447.36 | 0.511 | LST1 | SEQ ID NO: 89 | |
| M65254_at | 0.0007819 | 113.86 | 78.19 | 1.456 | PPP2R1B | SEQ ID NO: 90 | protein phosphatase 2 (formerly 2A), regulatory |
| | | | | | | | subunit A (PR 65), beta isoform |
| HG544-HT544_at | 0.0007893 | 839.34 | 2134.83 | 0.393 | ECGF1 | SEQ ID NO: 91 | Endothelial Cell Growth Factor 1 |
| L31584_at | 0.0007916 | 1975.87 | 800.47 | 2.468 | CCR7 | SEQ ID NO: 92 | |
| U09178_s_at | 0.0008134 | 219.29 | 323.01 | 0.679 | DPYD | SEQ ID NO: 93 | dihydropyrimidine dehydrogenase |
| D14874_at | 0.0008412 | 103.22 | 372.73 | 0.277 | ADM | SEQ ID NO: 94 | adrenomedullin |
| M74491_at | 0.0008619 | 2784.22 | 3977.01 | 0.7 | ARF3 | SEQ ID NO: 95 | ADP-ribosylation factor 3 |
| U57629_at | 0.0008653 | 54.83 | 88.79 | 0.618 | RPGR | SEQ ID NO: 96 | retinitis pigmentosa GTPase regulator |
| U89336_cds1_at | 0.0009043 | 1925.83 | 3119.22 | 0.617 | GPSM3 | SEQ ID NO: 97 | |
| M19961_at | 0.0009161 | 1222.72 | 1947.14 | 0.628 | COX5B | SEQ ID NO: 98 | cytochrome c oxidase subunit Vb |
| L29008_at | 0.0009279 | 164.98 | 228.84 | 0.721 | SORD | SEQ ID NO: 99 | sorbitol dehydrogenase |
| U16306_at | 0.0009612 | 2367.42 | 4644.34 | 0.51 | CSPG2 | SEQ ID NO: 100 | chondroitin sulfate proteoglycan 2 (versican) |
| S57212_s_at | 0.0009716 | 196.25 | 117.27 | 1.674 | MEF2C | SEQ ID NO: 101 | MADS box transcription enhancer factor 2, |
| | | | | | | | polypeptide C (myocyte enhancer factor 2C) |

TABLE 4
Genes with altered expression in patients with PAH compared to normal controls.

| | | | Increased or | | |
|---|---|---|---|---|---|
| GenBank | Gene | | Decreased in PAH | Chromosome | Sequence |
| ID number | symbol | Putative Function | vs. Normal | Location | Identifier |
| X60003 | CREB1 | Regulation of | Decreased in PAH | 2q34 | SEQ ID NO: 7 |
| | | transcription | | | |
| L31584 | CCR7 | Elevate cytosolic | Decreased in PAH | 17q12 | SEQ ID NO: 92 |
| | | calcium ion | | | |
| | | concentration | | | |
| M29696 | IL-7R | Antimicrobial humoral | Decreased in PAH | 5p13.2 | SEQ ID NO: 51 |
| | | response | | | |
| U32519 | G3BP | Ras protein signal | Decreased in PAH | 5q33.1 | SEQ ID NO: 54 |
| | | transduction | | | |
| U51478 | ATP1B3 | K+ ion transport | Increased in PAH | 3q22-q23 | SEQ ID NO: 6 |
| U67156 | MAP3K5 | Activation of JUNK, | Increased in PAH | 6q22 | SEQ ID NO: 9 |
| | | Induction of apoptosis | | | |
| X62320 | GRN | Growth factor | Increased in PAH | 17q21-32 | SEQ ID NO: 25 |
| L76191 | IRAK-1 | Defense response | Increased in PAH | Xq28 | SEQ ID NO: 29 |
| M32315 | TNFRSF1B | Apoptosis | Increased in PAH | 1p36.3 | SEQ ID NO: 50 |
| U03100 | CTNNA1 | Cell adhesion | Increased in PAH | 5q31 | SEQ ID NO: 62 |
| U09578 | MAPKAPK3 | Signal transduction, | Increased in PAH | 3p21.3 | SEQ ID NO: 65 |
| | | MAP kinase kinase | | | |
| | | activity | | | |
| L04270 | LTBR | Signal transduction, | Increased in PAH | 12p13 | SEQ ID NO: 67 |
| | | apoptosis | | | |
| M97935 | STAT1 | Regulation of | Increased in PAH | 2q32.2 | SEQ ID NO: 71 |
| | | transcription | | | |
| Z35491 | BAG1 | Anti-apoptosis | Increased in PAH | 9p12 | SEQ ID NO: 78 |
| U29680 | BCL2A1 | Anti-apoptosis | Increased in PAH | 15q24 | SEQ ID NO: 82 |
| M31932 | FCGR2a | Immune Response | Increased in PAH | 1q23 | SEQ ID NO: 83 |
| D30755 | TNIP1 | Negative regulation of | Increased in PAH | 5q32-q33.1 | SEQ ID NO: 19 |
| | | viral genome | | | |

TABLE 5

| | | | | Fold | | | |
|---|---|---|---|---|---|---|---|
| | | Mean | Mean | Change | | | |
| | Parametric | Intensity | Intensity | (IPAH/ | Gene | Sequence | |
| Probe set ID | P value | IPAH | s-PAH | s-PAH) | Symbol | Identifier | Gene Name |
| U70321_at | 4.77E−04 | 1206.174 | 1528.08 | 0.789 | TNFRSF14 | SED ID NO: 102 | tumor necrosis factor receptor superfamily, member |
| | | | | | | | 14 (herpesvirus entry mediator) |
| D42108_at | 1.14E−03 | 83.41 | 45.551 | 1.831 | PLCL1 | SED ID NO: 103 | phospholipase C-like 1 |
| D64154_at | 1.40E−03 | 654.873 | 906.069 | 0.723 | ADRM1 | SED ID NO: 104 | adhesion regulating molecule 1 |
| U33849_at | 1.49E−03 | 388.683 | 575.29 | 0.676 | PCSK7 | SED ID NO: 105 | proprotein convertase subtilisin/kexin type 7 |
| D86970_at | 2.26E−03 | 283.419 | 410.446 | 0.691 | TIAF1 | SED ID NO: 106 | TGFB1-induced anti-apoptotic factor 1 |
| M57763_at | 2.34E−03 | 636.291 | 1040.775 | 0.611 | ARF6 | SED ID NO: 107 | ADP-ribosylation factor 6 |
| M36542_s_at | 3.79E−03 | 295.638 | 615.272 | 0.48 | POU2F2 | SED ID NO: 108 | POU domain, class 2, transcription factor 2 |
| U10362_at | 4.22E−03 | 496.583 | 804.081 | 0.618 | LMAN2 | SED ID NO: 109 | chromosome 5 open reading frame 8 |
| U51477_at | 4.31E−03 | 1083.301 | 1429.71 | 0.758 | DGKZ | SED ID NO: 110 | diacylglycerol kinase, zeta 104 kDa |
| Y00486_rna1_at | 4.40E−03 | 1454.728 | 2194.127 | 0.663 | APRT | SED ID NO: 111 | adenine phosphoribosyltransferase |
| M24485_s_at | 5.41E−03 | 3129.692 | 3859.227 | 0.811 | GSTP1 | SED ID NO: 112 | glutathione S-transferase pi |
| D42053_at | 5.57E−03 | 572.646 | 825.123 | 0.694 | MBTPS1 | SED ID NO: 113 | membrane-bound transcription factor protease, site 1 |
| U80184_rna1_at | 5.72E−03 | 1172.307 | 1530.217 | 0.766 | FLII | SED ID NO: 84 | Homo sapiens FLII gene |
| M13194_at | 6.86E−03 | 328.579 | 422.288 | 0.778 | ERCC1 | SED ID NO: 114 | excision repair cross-complementing rodent repair |
| | | | | | | | deficiency, complementation group 1 |
| U64105_at | 7.24E−03 | 1449.56 | 1944.063 | 0.746 | ARHGEF1 | SED ID NO: 115 | Rho guanine nucleotide exchange factor (GEF) 1 |
| L15309_at | 7.32E−03 | 80.544 | 41.854 | 1.924 | ZNF141 | SED ID NO: 116 | zinc finger protein 141 (clone pHZ-44) |
| D79985_at | 7.58E−03 | 580.882 | 903.805 | 0.643 | DGCR2 | SED ID NO: 117 | DiGeorge syndrome critical region gene 2 |
| U82279_at | 8.26E−03 | 154.603 | 271.067 | 0.57 | LILRB1 | SED ID NO: 118 | leukocyte immunoglobulin-like receptor, subfamily |
| | | | | | | | B (with TM and ITIM domains), member 1 |
| U05572_s_at | 8.37E−03 | 1293.898 | 1783.139 | 0.726 | MAN2B1 | SED ID NO: 119 | mannosidase, alpha, class 2B, member 1 |
| L07044_at | 8.45E−03 | 366.275 | 502.032 | 0.73 | CAMK2G | SED ID NO: 120 | calcium/calmodulin-dependent protein kinase (CaM |
| | | | | | | | kinase) II gamma |
| L39059_at | 8.58E−03 | 135.579 | 185.025 | 0.733 | TAF1C | SED ID NO: 121 | TATA box binding protein (TBP)-associated factor, |
| | | | | | | | RNA polymerase I, C, 110 kDa |
| M74715_s_at | 8.64E−03 | 336.047 | 473.654 | 0.709 | IDUA | SED ID NO: 122 | iduronidase, alpha-L- |
| L19183_at | 9.35E−03 | 106.921 | 73.95 | 1.446 | MAC30 | SED ID NO: 123 | hypothetical protein MAC30 |
| X54637_at | 9.48E−03 | 814.664 | 1397.172 | 0.583 | TYK2 | SED ID NO: 124 | tyrosine kinase 2 |
| U07424_at | 9.54E−03 | 384.524 | 484.727 | 0.793 | FARSL | SED ID NO: 125 | phenylalanine-tRNA synthetase-like |
| L43631_at | 9.75E−03 | 811.139 | 1237.36 | 0.656 | SAFB | SED ID NO: 126 | scaffold attachment factor B |
| D87466_at | 9.85E−03 | 143.06 | 105.06 | 1.362 | KIAA0276 | SED ID NO: 127 | KIAA0276 protein |
| L36818_at | 9.95E−03 | 890.708 | 1218.665 | 0.731 | INPPL1 | SED ID NO: 128 | inositol polyphosphate-phosphatase-like 1 |

TABLE 6

| | | | | |
|---|---|---|---|---|
| | | Gen | | |
| | | Bank | | Fold |
| Gene Symbol | Gene Name | ID | P value | Change |

Inflammatory Response, z = 3.85

| | | | | |
|---|---|---|---|---|
| AGER | Advanced | U89336 | 0.000904 | 1.65 |
| | glycosylation | | | |
| | end product-specific | | | |
| | receptor | | | |
| ALOX5 | Arachidonate-5 | J03600 | 0.006021 | 1.79 |
| | Lipoxygensae | | | |
| ANXA1 | Annexin A1 | L19605 | 0.001932 | 1.41 |
| BCL6 | B-Cell CLL 6 | U00115 | 0.000497 | 2.347 |
| BDKRB2 | Bradykinin | X86163 | 0.018949 | 0.792 |
| | Receptor B2 | | | |
| CCR1 | Chemokine | D10925 | 0.013787 | 1.938 |
| | Receptor 1 | | | |
| CCR7 | Chemokine | L31584 | 0.000792 | 0.405 |
| | Receptor 7 | | | |
| CD14 | CD 14 antigen | D37781 | 0.470246 | 1.608 |
| CEBPB | CCAAAT/enhancer | X52560 | 0.001905 | 1.835 |
| | binding protein | | | |
| | (C/ebp), beta | | | |
| CXCL1 | Chemokine Ligand 1 | L36033 | 0.96005 | 3.268 |
| FPRL1 | Formyl Peptide | D10922 | 0.020683 | 2.141 |
| | Recptor-like 1 | | | |
| MYD88 | Myeloid | U70451 | 0.000056 | 1.59 |
| | Differentiation | | | |
| | Primary Response | | | |
| | Gene | | | |
| NMI | M-myc interactor | U32849 | 0.004111 | 1.46 |
| PTAFR | Platelet Acctivating | D10202 | 0.018205 | 1.54 |
| | Factor Receptor | | | |
| RAC1 | ras-related C-3 | D25274 | 0.111503 | 2.11 |
| | botulinum | | | |
| | Toxin Substrate | | | |
| S100A12 | S100 Calcium Binding | D83657 | 0.00171 | 2.26 |
| | Protein A12 | | | |
| S100A9 | S100 Calcium Binding | M26311 | 0.020869 | 1.59 |
| | Protein A9 | | | |
| TLR4 | Toll Like Receptor 4 | U93091 | 0.00256 | 2.43 |

Response to Stress, z = 3.31

| | | | | |
|---|---|---|---|---|
| MAPKAPK3 | Mitogen acctivated | U09578 | 0.000413 | 2.01 |
| | protein kinase | | | |
| AHR | Aryl hydrocarbon | L19872 | 0.008558 | 1.64 |
| | receptor | | | |
| HPK1 | Hematopoietic | U66464 | 0.01575 | 1.37 |
| | progenitor | | | |
| | kinase | | | |
| MAP3K5 | Mitogen acctivated | U67156 | 0.000015 | 2.16 |
| | protein kinase | | | |
| | kinase 5 | | | |
| MAP4K2 | Mitogen acctivated | U07349 | 0.015638 | 1.44 |
| | protein kinase kinase | | | |
| | kinase kinase 2 | | | |
| SUI1 | Putative trasnlation | L26247 | 0.008609 | 1.26 |
| | initiator factor | | | |

Cytochrome C Oxidase, z = 4.47

| | | | | |
|---|---|---|---|---|
| O95101 | Cytochrome C oxidase | X15341 | 0.004762 | 1.4 |
| | polypetide VIa-liver | | | |
| COX4I1 | Cytochrome C oxidase | U90915 | 0.002776 | 1.17 |
| | subunit IV precursor | | | |
| COX5A | Cytochrome C oxidase | M22760 | 0.001064 | 1.54 |
| | subunit Va | | | |
| COX5B | Cytochrome C oxidase | M19961 | 0.000916 | 1.59 |
| | subunit Vb | | | |
| COX6A1 | Cytochrome C oxidase | X15341 | 0.004767 | 1.4 |
| | subunit VIa | | | |
| | polypetide 1 | | | |
| COX7B | Cytochrome C oxidase | Z14244 | 0.016069 | 1.44 |
| | subunit VIIb | | | |
| COX8 | Cytochrome C oxidase | J04823 | 0.00146 | 1.42 |
| | subunit VIII | | | |

Lysosome, z = 5.73

| | | | | |
|---|---|---|---|---|
| ARSA | Aryl Sulfate A | U60276 | 0.588487 | 1.46 |
| ASAH1 | Acylsphingosine | U70063 | 0.00036 | 1.59 |
| | amidohydrolase 1 | | | |
| CTSB | Cathepsin B | L16510 | 0.000074 | 1.77 |
| CTSD | Cathepsin D | M63138 | 0.019057 | 1.36 |
| CTSL | Cathepsin L | X12451 | 0.004386 | 1.98 |
| GALC | Galactosylceramidase | D88667 | 0.691231 | 1.72 |
| GLA | Galactosidase, apha | D26443 | 0.300503 | 1.47 |
| HEXB | Hexosaminidase B | M23294 | 0.001771 | 1.54 |
| PPT2 | Patmitoyl-Protein | U89336 | 0.000904 | 1.62 |
| PSAP | Prosaposin | J03077 | 0.003034 | 1.4 |
| TIAL1 | TIA 1 cytotoxic | M96934 | 0.0237 | 1.34 |
| | granule-associated | | | |
| | ma binding protein- | | | |
| | like 1 | | | |
| CD63 | CD63 antigen | X62654 | 0.000354 | 1.99 |
| LAMP2 | Lysosomal associated | S79873 | 0.004052 | 1.66 |
| | membrane protein 2 | | | |

Intracellular Signaling Cascade, z = 3.16

| | | | | |
|---|---|---|---|---|
| ADCY7 | Adenylate cyclase 7 | D25538 | 0.025247 | 1.43 |
| BTK | Bruton | U78027 | 0.019268 | 1.47 |
| | agammaglobulinemia | | | |
| | tyrosine kinase | | | |
| CSK | C-SRC tyrosine kinase | X59932 | 0.001291 | 1.56 |
| CXCL1 | Chemokine (C-X-C | L36033 | 0.96005 | 3.268 |
| | motif) ligand | | | |
| FES | Feline sarcoma | X52192 | 0.007571 | 1.26 |
| | oncogene | | | |
| FGR | Gardner-Rasheed | M19722 | 0.002038 | 1.56 |
| | feline sarcoma viral | | | |
| | oncogens homolog | | | |
| IL16 | Interleukin 16 | M90391 | 0.055785 | 0.61 |
| | (lymphocye | | | |
| | chemoattractant factor) | | | |
| LIMK1 | Lim domain kinase 1 | U62293 | 0.016765 | 1.307 |

TABLE 7
Leave-one-out cross-validation performance
Performance of classifiers during cross-validation:

| | | | | Compound | Linear | | | | Support |
|---|---|---|---|---|---|---|---|---|---|
| | | | Number of | Covariate | Discriminant | 1-Nearest | 3-Nearest | Nearest | Vector |
| | | | genes in | Predictor | Analysis | Neighbor | Neighbors | Centroid | Machines |
| | Array id | Class label | classifier | Correct? | Correct? | Correct? | Correct? | Correct? | Correct? |
| 1 | 01_NOR | NOR | 84 | YES | YES | YES | YES | YES | YES |
| 2 | 02_NOR | NOR | 73 | YES | YES | YES | YES | YES | YES |
| 3 | 03_NOR | NOR | 71 | YES | YES | YES | YES | YES | YES |
| 4 | 04_NOR | NOR | 57 | YES | YES | YES | YES | YES | YES |
| 5 | 05_NOR | NOR | 95 | YES | YES | YES | YES | YES | YES |
| 6 | 06_NOR | NOR | 96 | YES | YES | YES | YES | YES | YES |
| 7 | 01_PAH | PAH | 88 | YES | YES | YES | YES | YES | YES |
| 8 | 14_PAH | PAH | 96 | YES | YES | YES | YES | YES | YES |
| 9 | 08_PAH | PAH | 93 | YES | YES | YES | YES | YES | YES |
| 10 | 02_PAH | PAH | 120 | YES | YES | YES | YES | YES | YES |
| 11 | 09_PAH | PAH | 96 | YES | YES | YES | YES | YES | YES |
| 12 | 10_PAH | PAH | 102 | YES | YES | YES | YES | YES | YES |
| 13 | 11_PAH | PAH | 94 | YES | YES | YES | YES | YES | YES |
| 14 | 15_PAH | PAH | 93 | YES | YES | YES | YES | YES | YES |
| 15 | 03_PAH | PAH | 108 | YES | YES | YES | YES | YES | YES |
| 16 | 12_PAH | PAH | 89 | YES | YES | YES | YES | YES | YES |
| 17 | 04_PAH | PAH | 111 | YES | YES | YES | YES | YES | YES |
| 18 | 13_PAH | PAH | 107 | YES | YES | YES | YES | YES | YES |
| 19 | 05_PAH | PAH | 109 | YES | YES | YES | YES | YES | YES |
| 20 | 06_PAH | PAH | 109 | YES | YES | YES | NO | NO | YES |
| 21 | 07_PAH | PAH | 96 | YES | YES | YES | YES | YES | YES |
| Percent | | | | 100 | 100 | 100 | 95 | 95 | 100 |
| correctly | | | | | | | | | |
| classified: | | | | | | | | | |

### REFERENCES

Each of the references cited below and elsewhere herein is incorporated by reference in its entirety.
1. Rubin, N. Engl. J. Med. 1997; 336: 111-7.
2. Bristow et al., Chest 1998; 114: 101S-6S.
3. Voelkel et al., Eur. Respir. J 1999; 14: 1246-50.
4. Rich, Curr. Treat. Options. Cardiovasc. Med. 2000; 2: 135-40.
5. Cool et al., Am. J. Pathol. 1999; 155: 411-9.
6. Tuder et al., Clin. Chest Med. 2001; 22: 405-18.
7. Tuder et al., American Journal of Pathology 1994; 144: 275-85.
8. Humbert et al., Clin. Chest Med. 2001; 22: 459-75.
9. Voelkel, Thorax 1997; 52 Suppl 3: S63-S67.
10. Dorfmuller et al., Eur. Respir. J. 2003; 22: 358-63.
11. Tuder et al., J. Lab Clin. Med. 1998; 132: 16-24.
12. Voelkel et al., Ann. N.Y. Acad. Sci. 1994; 725: 104-9.
13. Fagan et al., Prog. Cardiovasc. Dis. 2002; 45: 225-34.
14. Cool et al., Hum. Pathol. 1997; 28: 434-42.
15. Tuder et al., Am. J. Pathol. 1994; 144: 275-85.
16. Balabanian et al., Am. J. Respir. Crit Care Med. 2002; 165: 1419-25.
17. Dorfmuller et al., Am. J. Respir. Crit Care Med. 2002; 165: 534-9.
18. Lesprit et al., Am. J. Respir. Crit Care Med. 1998; 157: 907-11.
19. Humbert et al., Am. J. Respir. Crit Care Med. 1995; 151: 1628-31.
20. Isern et al., Am. J. Med. 1992; 93: 307-12.
21. Humbert et al., Eur. Respir. J. 1998; 11: 554-9.
22. Chu et al., Chest 2002; 122: 1668-73.
23. Bull et al., Eur Respir J 2003; 22: 403-7.
24. Cool et al., N. Engl. J. Med. 2003; 349: 1113-22.
25. Schena et al., Trends In Biotechnology 1998; 16: 301-6.
26. Khan et al., Nat. Med. 2001; 7: 673-9.
27. Bittner et al., Nature 2000; 406: 536-40.
28. Bhattacharjee et al., Proc. Natl. Acad. Sci. U.S.A 2001; 98: 13790-5.
29. Ramaswamy et al., Nat. Genet. 2003; 33: 49-54.
30. Dan et al., Cancer Res. 2002; 62: 1139-47.
31. Voelkel et al., Annals New York Academy of Sciences 1996; 796: 186-93.
32. Eddahibi et al., Am. J. Respir. Crit Care Med. 2000; 162: 1493-9.
33. Rus et al., Clin. Immunol. 2002; 102: 283-90.
34. Alcorta et al., Exp. Nephrol. 2002; 10: 139-49.
35. Gu et al., Rheumatology (Oxford) 2002; 41: 759-66.
36. Koike et al., J. Neuroimmunol. 2003; 139: 109-18.
37. Whitney et al., Proc. Natl. Acad. Sci. U.S.A 2003; 100: 1896-901.
38. Geraci et al., Circ. Res. 2001; 88: 555-62.
39. Golub et al., Science 1999; 286: 531-7.
40. Brown et al., Nature Genetics 1999; 21: 33-7.
41. Alizadeh et al., Nature 2000; 403: 503-11.
42. 't Veer et al., Nature 2002; 415: 530-6.
43. Huang et al., Arthritis Rheum. 2002; 47: 249-54.
44. Ramanathan et al., J. Neuroimmunol. 2001; 116: 213-9.
45. Thompson and McRae, Br. Heart J. 1970; 32: 758-60.
46. Loyd et al., Am. J. Respir. Crit Care Med. 1995; 152: 93-7.
47. Deng et al., Am. J. Hum. Genet. 2000; 67: 737-44.
48. De Caestecker et al., Respir. Res. 2001; 2: 193-7.
49. Balabanian et al., Am. J. Respir. Crit Care Med. 2002; 165: 1419-25.
50. Simon et al., J. Natl. Cancer Inst. 2003; 95: 14-8.
51. Liu et al., Cell 1990; 61: 1217-24.
52. Maekawa et al., Journal of Biological Chemistry 1999; 274: 17813-9.
53. Ronai et al., Oncogene 1998; 16: 523-31.
54. Falvo et al., Mol. Cell Biol. 2000; 20: 4814-25.
55. Monzen et al., J. Cell Biol. 2001; 153: 687-98.
56. Sano et al., J. Biol. Chem. 1999; 274: 8949-57.
57. Montgomery et al., Cell 1996; 87: 427-36.
58. Yoshibayashi et al., Am. J. Cardiol. 1997, 79:1556-1558.
59. Lippton et al., J Appl. Physiol 1994, 76:2154-2156.
60. Kakishita et al., Clin Sci. (Lond) 1999, 96:33-39.
61. Kandler et al., J Pharmacol. Exp Ther. 2003, 306:1021-1026.
62. Ishikawa et al., Nature 1989, 338:557-562.
63. Moghaddam et al., Proc. Natl. Acad. Sci. U.S.A 1995, 92:998-1002.
64. Reynolds et al., J Natl. Cancer Inst. 1994, 86:1234-1238.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. A method to diagnose pulmonary arterial hypertension (PAH) or a predisposition to develop PAH, comprising:
a) detecting in a sample of peripheral blood cells from a patient to be tested the level of expression of at least one biomarker chosen from a panel of biomarkers whose expression in peripheral blood cells has been associated with PAH as measured by either upregulation or downregulation of biomarker expression in peripheral blood cells from patients with PAH as compared to the level of expression of the biomarkers in peripheral blood cells from normal controls;
b) comparing the level of expression of the biomarker or biomarkers detected in the patient sample to a level of expression of the biomarker or biomarkers that has been associated with PAH and a level of expression of the biomarker or biomarkers that has been associated with normal controls; and
c) diagnosing PAH in the patient if the expression level of the biomarker or biomarkers in the patient sample is statistically more similar to the expression level of the biomarker or biomarkers that has been associated with PAH than the expression level of the biomarker or biomarkers that has been associated with the normal controls.

2. The method of claim 1, wherein the panel of biomarkers in (a) is identified by a method comprising;
a) comparing the expression level of at least one biomarker in peripheral blood cells from patients that have PAH to the level of expression of the biomarker in peripheral blood cells from normal controls that do not have PAH; and
b) identifying a biomarker or biomarkers having a level of expression in peripheral blood cells from patients with PAH that is statistically significantly different than the level of expression of the biomarker or biomarkers in the peripheral blood cells from the normal controls, as being a biomarker for use in a panel of biomarkers to diagnose PAH.

3. The method of claim 1, wherein step (a) comprises detecting in the patient sample the expression of at least one gene chosen from a gene comprising, or expressing a transcript comprising, a nucleic acid sequence selected from the group consisting of SEQ ID NOs:1-101;
wherein step (b) comprises comparing the level of expression of the gene or genes detected in the patient sample to a level of expression of the gene or genes that has been associated with PAH and to a level of expression of the gene or genes that has been associated with normal controls; and
wherein step (c) comprises diagnosing PAH in the patient, if the expression of the gene or genes in the patient sample is statistically more similar to the expression level of the gene or genes that has been associated with PAH than with normal controls.

4. The method of claim 3, wherein the step (a) of detecting comprises detecting expression of at least 2 genes.

5. The method of claim 3, wherein the step (a) of detecting comprises detecting expression of at least 5 genes.

6. The method of claim 3, wherein the step (a) of detecting comprises detecting expression of at least 10 genes.

7. The method of claim 3, wherein the step (a) of detecting comprises detecting expression of at least 25 genes.

8. The method of claim 3, wherein the step (a) of detecting comprises detecting expression of at least 50 genes.

9. The method of claim 3, wherein the step (a) of detecting comprises detecting expression of at least 75 genes.

10. The method of claim 3, wherein the step (a) of detecting comprises detecting expression of at least 100 genes.

11. The method of claim 3, wherein the step (a) of detecting comprises detecting expression of at least 125 genes.

12. The method of claim 3, wherein the step (a) of detecting comprises detecting expression of each of SEQ ID NOs:1-101.

13. The method of claim 3, wherein expression of the gene or genes is detected by measuring amounts of transcripts of the gene in the patient peripheral blood cells.

14. The method of claim 3, wherein expression of the gene or genes is detected by detecting hybridization of at least a portion of the gene or a transcript thereof to a nucleic acid molecule comprising a portion of the gene or a transcript thereof in a nucleic acid array.

15. The method of claim 3, wherein expression of the gene or genes is detected using quantitative polymerase chain reaction (q-PCR).

16. The method of claim 3, wherein expression of the gene is detected by detecting the production of a protein encoded by the gene.

17. The method of claim 3, further comprising determining if the patient has idiopathic pulmonary arterial hypertension (IPAH) or secondary pulmonary arterial hypertension (s-PAH), said step of determining comprising:
a) comparing the level of expression of at least one gene chosen from a gene comprising, or expressing a transcript comprising, a nucleic acid sequence selected from the group consisting of SEQ ID NO:84, SEQ ID NOs:102-128;
b) comparing the level of expression of the gene or genes detected in the patient sample to a level of expression of the gene or genes that has been associated with IPAH and to a level of expression of the gene or genes that has been associated with s-PAH; and
c) diagnosing IPAH in the patient, if the expression of the gene or genes in the patient sample is statistically more similar to the expression level of the gene or genes that has been associated with IPAH than with s-PAH, or diagnosing s-PAH in the patient, if the expression of the gene or genes in the patient sample is statistically more similar to the expression level of the gene or genes that has been associated with s-PAH than with IPAH.

18. The method of claim 3, wherein the level of expression of the gene or genes that has been associated with PAH and the level of expression of the gene or genes that has been associated with normal controls has been predetermined.

19. A plurality of polynucleotides for the detection of the expression of genes that indicate a diagnosis of pulmonary arterial hypertension (PAH) in a patient, wherein the plurality of polynucleotides consists of at least two polynucleotides, wherein each polynucleotide is at least 5 nucleotides in length, and wherein each polynucleotide is complementary to an RNA transcript, or nucleotide derived therefrom, of a gene that is regulated differently in peripheral blood cells of patients with PAH as compared to peripheral blood cells of individuals that do not have PAH.

20. The plurality of polynucleotides of claim 19, wherein each polynucleotide is complementary to an RNA transcript, or a polynucleotide derived therefrom, of a gene comprising, or expressing a transcript comprising, a nucleic acid sequence selected from the group consisting of SEQ ID NOs:1-128.

21. The plurality of polynucleotides of claim 19, wherein the plurality of polynucleotides comprises polynucleotides that are complementary to an RNA transcript, or a nucleotide derived therefrom, of at least two genes comprising, or expressing a transcript comprising, a nucleic acid sequence selected from the group consisting of SEQ ID NOs:1-128.

22. The plurality of polynucleotides of claim 19, wherein the plurality of polynucleotides comprises polynucleotides that are complementary to an RNA transcript, or a nucleotide derived therefrom, of at least five genes comprising, or expressing a transcript comprising, a nucleic acid sequence selected from the group consisting of SEQ ID NOs:1-128.

23. The plurality of polynucleotides of claim 19, wherein the plurality of polynucleotides comprises polynucleotides that are complementary to an RNA transcript, or a nucleotide derived therefrom, of at least 10 genes comprising, or expressing a transcript comprising, a nucleic acid sequence selected from the group consisting of SEQ ID NOs:1-128.

24. The plurality of polynucleotides of claim 19, wherein the plurality of polynucleotides comprises polynucleotides that are complementary to an RNA transcript, or a nucleotide derived therefrom, of at least 25 genes comprising, or expressing a transcript comprising, a nucleic acid sequence selected from the group consisting of SEQ ID NOs:1-128.

25. The plurality of polynucleotides of claim 19, wherein the plurality of polynucleotides comprises polynucleotides that are complementary to an RNA transcript, or a nucleotide derived therefrom, of at least 50 genes comprising, or expressing a transcript comprising, a nucleic acid sequence selected from the group consisting of SEQ ID NOs:1-128.

26. The plurality of polynucleotides of claim 19, wherein the plurality of polynucleotides comprises polynucleotides that are complementary to an RNA transcript, or a nucleotide derived therefrom, of at least 100 genes comprising, or expressing a transcript comprising, a nucleic acid sequence selected from the group consisting of SEQ ID NOs:1-128.

27. The plurality of polynucleotides of claim 19, wherein the plurality of polynucleotides comprises polynucleotides that are complementary to an RNA transcript, or a nucleotide derived therefrom, of all of the genes comprising, or expressing a transcript comprising, a nucleic acid sequence selected from the group consisting of SEQ ID NOs:1-128.

28. The plurality of polynucleotides of any one of claims 19, wherein said polynucleotide probes are immobilized on a substrate.

29. The plurality of polynucleotides of any one of claims 19, wherein said polynucleotide probes are hybridizable array elements in a microarray.

30. The plurality of polynucleotides of any one of claims 19, wherein said polynucleotide probes are conjugated to detectable markers.

31. A method to monitor the treatment of a patient with pulmonary arterial hypertension (PAH), comprising:
a) detecting the level of expression of at least one gene in a sample of peripheral blood cells isolated from a patient undergoing treatment for PAH, wherein the gene is chosen from a gene comprising, or expressing a transcript comprising, a nucleic acid sequence selected from the group consisting of SEQ ID NOs:1-101; and
b) comparing the level of expression of comparing the level of expression of the gene or genes detected in the patient sample to the level of expression of the gene or genes in a prior sample of peripheral blood cells from the patient and to a level of expression of the gene or genes in peripheral blood cells from normal controls that do not have PAH, wherein detection of a change in the level of expression of the gene or genes, as compared to the level of expression in the prior sample, toward the level of the expression of the gene in a normal control sample, indicates that the treatment for pulmonary hypertension is producing a beneficial result.

32. A method to diagnose a pulmonary disease or condition in a patient, comprising:
a) detecting in a sample of peripheral blood cells from a patient to be tested the level of expression of at least one biomarker chosen from a panel of biomarkers whose expression in peripheral blood cells has been associated with a pulmonary disease as measured by either upregulation or downregulation of biomarker expression in peripheral blood cells from patients with the pulmonary disease as compared to the level of expression of the biomarkers in peripheral blood cells from normal controls that do not have the pulmonary disease;
b) comparing the level of expression of the biomarker or biomarkers detected in the patient sample to a level of expression of the biomarker or biomarkers that has been associated with the pulmonary disease and a level of expression of the biomarker or biomarkers that has been associated with normal controls; and
c) diagnosing the pulmonary disease in the patient if the expression level of the biomarker or biomarkers in the patient sample is statistically more similar to the expression level of the biomarker or biomarkers that has been associated with the pulmonary disease than the expression level of the biomarker or biomarkers that has been associated with the normal controls.

33. The method of claim 32, wherein the disease or condition is a heart disease.

34. A method to identify a compound with the potential to treat pulmonary arterial hypertension (PAH), comprising:
a) contacting a test compound with a cell that expresses a gene chosen from a gene comprising, or expressing a transcript comprising, a nucleic acid sequence selected from the group consisting of SEQ ID NOs:1-128; and
b) identifying compounds that:
i) increase the expression or activity of the gene or protein encoded thereby if the expression of the gene is downregulated in peripheral blood cells of patients with pulmonary arterial hypertension as compared to the expression or activity of the gene or encoded protein in peripheral blood cells of normal controls; or
ii) decrease the expression or activity of the gene or protein encoded thereby if the expression of the gene is upregulated in peripheral blood cells of patients with pulmonary arterial hypertension as compared to the expression or activity of the gene or encoded protein in peripheral blood cells of normal controls.

35. The method of claim 34, wherein the cell expresses a nucleic acid molecule (represented by SEQ ID NO:94) encoding adrenomedullin, and wherein step (b) comprises identifying compounds that decrease the expression or activity of adrenomedullin or the gene encoding adrenomedullin.

36. The method of claim 34, wherein the cell expresses a nucleic acid molecule (represented by SEQ ID NO:91) encoding endothelial cell growth factor-1, and wherein step (b) comprises identifying compounds that decrease the expression or activity of endothelial cell growth factor-1 or the gene encoding endothelial cell growth factor-1.
